(19) United States
(12) Patent Application Publication
(10) Pub. No.: US 2025/0288808 A1
(43) Pub. Date: Sep. 18, 2025

(54) TREATMENT OF MULTIPLE SCLEROSIS

(71) Applicant: SetPoint Medical Corporation, Valencia, CA (US)

(72) Inventors: Jacob A. LEVINE, West Hempstead, NY (US); Nicole HAMLIN, Centereach, NY (US); David CHERNOFF, Sausalito, CA (US); Chandramohan NATARAJAN, Valencia, CA (US); Linh H.D. LE, Valencia, CA (US)

(21) Appl. No.: 19/224,699

(22) Filed: May 30, 2025

Related U.S. Application Data
(63) Continuation-in-part of application No. 18/664,817, filed on May 15, 2024.
Application No. 18/664,817 is a continuation-in-part of application No. 16/158,222, filed on Oct. 11, 2018, now Pat. No. 12,172,017.
(60) Provisional application No. 62/572,374, filed on Oct. 13, 2017.
(60) Provisional application No. 62/576,547, filed on Oct. 24, 2017.
(60) Provisional application No. 63/654,006, filed on May 30, 2024.

(51) Int. Cl. A61N 1/36

(52) U.S. Cl. CPC A61N 1/36103; A61N 1/36053; A61N 1/36139

(57) ABSTRACT

Method for reversing demyelination in a patient having a demyelinating disorder may include detecting one or more biomarkers for demyelination from the patient, receiving, in an implanted vagus nerve stimulator, a command to apply a remyelinating stimulation to the patient based on a level of the detected one or more biomarkers for demyelination; and applying electrical stimulation to the patient's vagus nerve from the implanted vagus nerve stimulator to reverse demyelination and increase remyelination of the patient's nerves. The electrical stimulation may be adjusted based on the level of the detected one or more biomarkers.

### CROSS REFERENCE TO RELATED APPLICATIONS

This patent application is a continuation-in-part of U.S. patent application Ser. No. 18/664,817, titled “REVERSING DEMYELINATION,” filed on May 15, 2024, now U.S. Patent Application Publication No. 2024/0299745, which claims priority as a continuation-in-part of U.S. patent application Ser. No. 16/158,222, titled “VAGUS NERVE STIMULATION TO TREAT NEURODEGENERATIVE DISORDERS,” filed on Oct. 11, 2018, now U.S. Pat. No. 12,172,017, which claims priority to U.S. Provisional Patent Application No. 62/572,374, filed on Oct. 13, 2017 (titled “VAGUS NERVE STIMULATION TO TREAT NEURODEGENERATIVE DISORDERS”) and U.S. Provisional Patent Application No. 62/576,547, filed Oct. 24, 2017 (titled “VAGUS NERVE STIMULATION TO TREAT NEURODEGENERATIVE DISORDERS”) each of which is herein incorporated by reference in its entirety.

This patent application also claims the benefit of U.S. Provisional Patent Application No. 63/654,006, filed on May 30, 2024, titled “ELECTRICAL STIMULATION OF THE VAGUS NERVE AMELIORATES INFLAMMATION AND DISEASE ACTIVITY IN A RAT EAE MODEL OF MULTIPLE SCLEROSIS,” which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the invention relate generally to apparatuses (e.g., devices, systems) and methods for vagus nerve stimulation to treat neurodegenerative and neuroinflammatory disorders, and more specifically apparatuses and methods for vagus nerve stimulation to reduce demyelination and/or to promote remyelination to treat various neurodegenerative disorders such as multiple sclerosis.

### BACKGROUND

A variety of central nervous system (CNS) demyelinating disorders, including multiple sclerosis, acute disseminated encephalomyelitis and neuromyelitis optica spectrum disorders, are difficult to effectively treat. For example, multiple sclerosis (MS) is a neurodegenerative disease characterized by demyelination of nerves in the central nervous system. Although the root cause of demyelination is not well understood, it generally is associated with the formation of lesions on the myelin sheaths and inflammation. Currently, there is no known cure for MS. Current treatments, with modest success, are primarily directed to treating acute attacks and reducing the frequency of attacks in the relapsing-remitting subtype of the disease or treating the symptoms. However, current therapies at best only slow the progression of the disease, and no therapy to date has demonstrated an ability to remyelinate nerves.

Therefore, it would be desirable to provide additional treatment methods and systems that can be used independently or in conjunction with other therapies to reduce the rate or amount of demyelination. Furthermore, it would be desirable to provide a therapy that remyelinates nerves and reverses the progression demyelination. In addition, it would be desirable to reduce inflammation in the nervous system.

MS is the most common disabling neurologic disease in young adults, and results in demyelination within the central nervous system, decrements in functional ability, and progressive accumulation of disability. Electrical stimulation of the vagus nerve (VNS) has been shown to provide clinical benefits for disorders including rheumatoid arthritis, epilepsy, depression, and Crohn's disease. There is a need for therapies, particularly non-drug therapies, to treat MS. The methods and apparatuses described herein may address these needs.

### SUMMARY OF THE DISCLOSURE

The present invention relates generally to vagus nerve stimulation to treat neurodegenerative disorders, and more specifically to vagus nerve stimulation to reduce demyelination and/or to promote remyelination to treat a demyelinating disorder. For example, described herein are method of reversing demyelination in a patient having a demyelinating disorder, the method comprising: receiving, in an implanted vagus nerve stimulator, a command to apply a remyelinating stimulation to the patient based on one or more biomarkers for demyelination; and applying electrical stimulation of between about 0.25 mA and about 5 mA at a duty cycle of less than 10 percent to the patient's vagus nerve from the implanted vagus nerve stimulator to reverse demyelination and increase remyelination of the patient's nerves.

In some cases applying comprises applying the electrical stimulation, followed by an off-time of at least 10 minutes. For example, applying may comprise applying the electrical stimulation for less than about 2 minutes. In some cases applying comprises applying the electrical stimulation for less than about 2 minutes, followed by an off-time of at least 10 minutes.

Monitoring may be performed continuously or discretely, and/or acutely. For example, monitoring of one or more biomarkers for demyelination may include continuously monitoring the patient for the one or more biomarkers for demyelination (e.g., sampling the patient at a frequency of 0.1 Hz or greater, 1 Hz or greater, 10 Hz or greater, etc.). Any of these methods may include detecting the one or more biomarkers for demyelination. Examples of biomarkers that may be monitored may include monitoring the patient's temperature, determining a level of tumor necrosis factor in the patient's blood or cerebrospinal fluid, etc. In some cases the one or more biomarkers for demyelination may be selected from the group consisting of neurofilament, glial fibrillary acidic protein, the monocyte macrophage marker CD163, the glial activation marker YKL-40, the B cell chemoattractant CXCL13, miRNA, mRNA, myelin reactive t cells, Kir4.1 antibodies, osteopontin, and microbiome associated lipopeptides.

The dose of electrical energy may be adjusted based on the level of the biomarker detected. For example, the application of the dose and/or the current amplitude and/or charge density of the dose applied may be adjusted based on the level of the one or more biomarkers relative to a threshold. The threshold may be based on a level that is correlated to the percentage or rapidity of demyelination. For example, a threshold for applying the remyelinating dose may be based on a percentage relative to an average value for the patient and/or a population that is similar (based on age, weight, gender, etc., to the patient), e.g., greater than 5%, greater than 10%, greater than 15%, greater than 20%, greater than 25%, greater than 30%, etc.

In some cases the dose may be repeated, e.g., for a therapeutically effective period of time. In some examples, the method may include repeatedly applying a low duty-cycle electrical stimulation of between about 0.25 and about 5 mA to the patient's vagus nerve for less than 2 minutes, followed by an off-time of between about 12 and about 48 hours.

For example, a method of reversing demyelination in a patient having a demyelinating disorder may include: detecting one or more biomarkers for demyelination from the patient; receiving, in an implanted vagus nerve stimulator, a command to apply a remyelinating stimulation to the patient based on a level of the detected one or more biomarkers for demyelination; and applying electrical stimulation of between about 0.25 mA and about 5 mA at a duty cycle of less than 10 percent to the patient's vagus nerve from the implanted vagus nerve stimulator to reverse demyelination and increase remyelination of the patient's nerves.

As mentioned above, applying may comprise applying the electrical stimulation, followed by an off-time of at least 10 minutes. Applying may comprise applying the electrical stimulation for less than about 2 minutes. Applying may comprise applying the electrical stimulation for less than about 2 minutes, followed by an off-time of at least 10 minutes.

The demyelinating disorder may be any disorder that results in acute or chronic demyelination of all or some of the patient's nerves. For example a demyelinating disorder may include neurodegenerative disorders such as but not limited to multiple sclerosis.

Although it has been suggested that vagus nerve stimulation may be used to treat inflammation, the application of electrical stimulation to the vagus nerve as described herein may result in remyelination even where inflammation is not associated with the disorder. Furthermore, the application of electrical stimulation to the vagus does not always result in an increase in myelination of nerves, even in patients suffering from demyelination disorders having an inflammatory component (such as multiple sclerosis). The methods described herein may be particularly useful in patients in which active demyelination is occurring. Thus, in some cases, the timing of the application of electrical energy and/or the dose of electrical energy may be coordinated with the detection of one or more biomarkers for demyelination, as described above. In some cases the electrical stimulation may be independent of the inflammatory status of the patient.

Also described herein are apparatuses (e.g., systems, devices, etc., including neurostimulators) for performing any of these methods.

For example, described herein are apparatuses (e.g., devices and/or systems) for reducing demyelination and/or increase remyelination by stimulation of a vagus nerve. These apparatuses may be implants or implanted into the patient's body. Any of these apparatuses may include: a biosensor configured to detect one or more biomarkers; a stimulator configured to apply stimulation to the vagus nerve; and a controller coupled to the biosensor and the stimulator and configured to apply stimulation to the vagus nerve from the stimulator sufficient to reduce demyelination and/or increase remyelination of nerves within the patient when the biosensor detects a biomarker indicative of demyelination. In some variations, these apparatuses include an implant comprising a stimulator (e.g., a waveform and/or pulse generator, an oscillator, a power supply and/or power regulation circuit, etc.), a stimulation applicator (e.g., one or more electrodes, mechanical transducers, etc.), and a controller. The controller may be configured as a microcontroller and may be in electrical communication with the stimulator so as to control operation of the stimulator. The controller may include one or more processors, a memory and/or a timer. The stimulator and/or controller may be in electrical communication, one or more stimulation applicators. In some variations the controller may include or be in communication with wireless communications circuitry for wirelessly communicating with one or more remote processors. The remote processor may be a hand-held device (e.g., smartphone, wearable electronics, etc.). The controller may optionally be in communication with one or more biosensors that may be included with the implant or may be remote from the implant (e.g., may be wearable, single-use, etc.). In some variations the biosensors are wirelessly connected to the apparatus.

In some variations the apparatus may be used without a biosensor. For example, the apparatus may be configured to periodically and/or on demand apply VNS treatment to prevent or reduce demyelination. The apparatus may be configured to apply VNS treatment doses once multiple times per day (e.g., 1× day, 2×, day, 3×, day, 4× day, 5× day, 6× day), or every other day, or every 3 days, etc. In some variations the apparatus may be configured to both automatically apply a VNS treatment dose on a predetermined and/or adjustable scheduled, as well as provide VNS treatment doses based on input from a user (e.g., patient, physician, etc., including “on demand” doses) and/or based on detection of a biomarker indicative of an actual or potential increase in demyelination.

In any of these variations, a biosensor may be configured to detect one or more markers (e.g., biomarkers) from the patient's body, including from the patient's blood and/or cerebrospinal fluid. Examples of biomarkers may be found herein. The biosensor may be part of the implanted apparatus, or it may be connected to the apparatus (e.g., the controller) via a wired or wireless communication. The biosensor may be configured to detect any biological marker, including chemical markers (e.g., a protein, nucleotide, e.g., RNA, DNA, microRNA, etc., lipid, carbohydrate, etc.), as well as functional markers (nerve conduction, etc.), body temperature, and the like. For example, in some variations, the biosensor is configured to detect temperature.

In general, the apparatuses described herein may be configured to be inserted or implanted into the body. For example, the apparatus may be configured to be implanted. The apparatus may include a stimulation applicator (also referred to as simply a stimulator or a VNS treatment stimulator) that may be a mechanical and/or electrical stimulator. A mechanical stimulator may be a piezoelectric driver that may vibrate and/or apply pressure to the tissue, including to the vagus nerve, in the VNS treatment parameters, such as mechanical stimulation of the vagus nerve at between 1-2 kHz for a treatment time (e.g., between 1 ms and 5 minutes, e.g., 10 ms-10 sec, etc.). Alternatively or additionally, the stimulation applicator may be an electrical stimulation applicator and may include one or more (e.g., two or more) electrodes configured to apply electrical stimulation to the vagus nerve. For example, electrical stimulation of about 0.1 mA to 10 mA (e.g., between 1 mA-5 mA), at a frequency of between about 1 Hz and about 2 kHz (e.g., between about 1-100 Hz), where the pulses applied have a pulse width of between about (50-500 usec, e.g., between about 100-300 usec). The controller may be configured to enforce an ‘off-time’ following a VNS treatment dose of between about 10 minute and 12 hours (e.g., between about 2 hours and 10 hours, between about 3 hours and 6 hours, at least 2 hours, at least 3 hours, at least 4 hours, at least 5 hours, at least 6 hours, etc.). For example, the stimulator may include an electrode configured to apply electrical energy to the vagus nerve.

In some variation the apparatus is configured to apply VNS treatment to the patient in which the VNS treatment is electrical stimulation. For example, the VNS treatment may include the application of electrical energy at between about 1-100 Hz (e.g., between about 1-50 Hz, between about 1-20 Hz, between about 5-30 Hz, between about 5-15 Hz, approximately 5 Hz, approximately 10 Hz, approximately 15 Hz, etc.). The energy may have a peak amplitude of between about 0.1 mA and about 2 mA (e.g., between about 0.2 mA and about 1.8 mA, between about 0.5 mA and about 1.5 mA, between about 0.5 mA and about 1 mA, between about 0.1 mA and about 1 mA, approximately 0.5 mA, approximately 0.75 mA, approximately 1 mA, etc.). Alternatively the applied energy may have an average amplitude of between about 0.1 mA and about 2 mA (e.g., between about 0.2 mA and about 1.8 mA, between about 0.5 mA and about 1.5 mA, between about 0.5 mA and about 1 mA, between about 0.1 mA and about 1 mA, approximately 0.5 mA, approximately 0.75 mA, approximately 1 mA, etc.). The applied energy is typically pulsed, and may be pulsed square waves, sinusoidal waves, triangular waves, etc. The applied energy may be biphasic or monophasic. For example, the applied energy may be biphasic. The applied VNS treatment may be a constant biphasic pulse train having a frequency of between 1-100 Hz (e.g., 10 Hz) and a peak amplitude of between about 0.5 mA and 2 mA (e.g., approximately 0.75 mA). Any of the methods for treatment described herein may be configured to apply this type of VNS treatment.

Any of the apparatuses (e.g., devices, systems, etc.) described herein may be configured to be implanted on the vagus nerve. Thus, any of these apparatuses may be implanted via a nerve sheath or nerve cuff configured to secure the apparatus onto the nerve and/or prevent movement of the apparatus relative to the nerve and/or insulate the apparatus from other tissues. The implanted apparatus may be implanted in any appropriate location on the nerve, including one or around the vagus nerve at the upper chest, or on or around the vagus nerve at a sub-diaphragmatic location. The implant may be a leadless implant that is connected to the vagus (see, e.g., U.S. Pat. Nos. 8,412,338, 8,612,002, 8,886,339, and 8,788,034, each of which is herein incorporated by reference in its entirety). For example, any of these apparatuses may include a nerve cuff configured to secure the stimulator to the vagus nerve. Alternatively, any of these apparatuses may include a lead connecting the micro stimulator and/or other components to the stimulation applicator on/around the vagus nerve via one or more leads.

As mentioned, any of these apparatuses may be configured to apply VNS treatment comprising a low duty-cycle electrical stimulation of between about 0.25 mA and about 5 mA to the vagus nerve for less than about 2 minutes. The apparatus may be configured to provide an off-time of at least x minutes/hours (e.g., 10 minutes, 20 minutes, 30 minutes, 40 minutes, 60 minutes, 2 hours, 3 hours, 4 hours, etc.).

Any of the apparatuses described herein may be configured to perform a method of reducing demyelination in a patient diagnosed with or at risk of a disorder involving demyelinated nerves (e.g., including but not limited to methods of treating a disorder and/or disease associated with demyelination, such as multiple sclerosis). For example, a method of reducing demyelination (and/or a method of increasing remyelination) may be a method comprising detecting a marker for demyelination and applying stimulation to the vagus nerve to reduce demyelination of nerves within the patent.

Applying stimulation to the vagus nerve includes applying VNS treatment and may comprise, for example, applying electrical stimulation of between about 0.25 and about 5 mA to the vagus nerve for less than about 2 minutes. In some variations this may include waiting for an off-time (e.g., an off-time of at least 10 minutes, 30 minutes, 1 hour, 2 hours, 3 hours, 4 hours, etc.).

Any of these methods may include applying non-invasive stimulation to the vagus nerve. For example, the simulation may be through a transdermal (e.g., via a surface electrode and/or mechanical stimulation, including ultrasound) route over a portion of the vagus nerve. The vagus nerve includes a number of branches or extensions that may be accessed and/or targeted from outside of the body either mechanically and/or electrically. For example, non-invasive applications may include ultrasound stimulation of the vagus nerve. Any of these methods may include applying transdermal electrical stimulation (TENS), or the like.

Any of the methods described herein may include monitoring, e.g., periodically, on demand, and/or continuously, one or more markers (e.g., biomarkers) for demyelination or a risk of demyelination. As mentioned, any appropriate method or apparatus for monitoring demyelination or a risk of demyelination may be used. For example any of these methods may include detecting a marker for demyelination comprising monitoring the patient's temperature. A change (including an increase) in core body temperature has been linked to an increase in symptoms in demyelination disorders, including but not limited to MS.

Any of the methods and apparatuses described herein may be used with or linked to markers for the integrity of the blood-brain barrier. The methods and apparatuses described herein generally improve the integrity of the blood-brain barrier. Thus, any marker linked to leakage or loss of integrity of the blood-brain barrier may be used to trigger VNS therapy as described herein. Examples of markers may include Serum S1008, as well as imaging modalities such as contrast-enhanced magnetic resonance imaging, CT-scan and lumbar puncture.

A detection of one or more markers (e.g., biomarkers) for demyelination may include determining a level of tumor necrosis factor in a blood or cerebrospinal fluid sample.

For example, described herein are methods (e.g., methods of treating a demyelination disorder, such as but not limited to MS, and/or methods of reducing or reversing demyelination) that include: detecting demyelination in a patient, and applying stimulation to the vagus nerve to increase the remyelination of nerves within the patent.

For example, any of these methods may include repeatedly applying a low duty-cycle electrical stimulation of between about 0.25 and about 5 mA to the patient's vagus nerve for less than about 2 minutes, followed by an off-time (e.g., of between about 10 minutes and about 48 hours) before the next stimulation.

Any of these methods and apparatuses may also include or be adapted to include the concurrent (immediately before, during or after, including systemically and/or locally) treatment with one or more pharmacological agents, particularly those that are believed to help with a demyelinating condition, such as (but not limited to) MS. For example, any of these method may include concurrently treating with a pharmacological agent such as one or more of: interferon beta-la, interferon beta-1b, glatiramer acetate, glatiramer acetate, peginterferon beta-la, daclizumab, teriflunomide, fingolimod, dimethyl fumarate, alemtuzumab, mitoxantrone, ocrelizumab, natalizumab.

As mentioned, any of the methods and apparatuses described herein may include continuously monitoring the patient for demyelination or a condition implicated in demyelination. For example, any of these methods and apparatuses described herein may include monitoring the patient for a marker related to a diseased selected from the group consisting of neurodegenerative diseases, neuroinflammatory diseases, and neuropathies. In some examples, the method includes detecting demyelination in a patient by detecting a marker related to MS. For example, the marker (e.g., biomarker) may be selected from the group including: neurofilament, glial fibrillary acidic protein, the monocyte macrophage marker CD163, the glial activation marker YKL-40, the B cell chemoattractant CXCL13, miRNA, mRNA, myelin reactive t cells, Kir4.1 antibodies, osteopontin, and microbiome associated lipopeptides.

The methods and apparatuses described herein demonstrate, for the first time, that VNS decreases both clinical symptoms and molecular pathology in experimental autoimmune encephalomyelitis (EAE), a standard rat model of MS. Surprisingly, VNS modulates gene expression, including those encoding inflammatory mediators, inflammatory reflex components, and oligodendrocyte differentiation and myelin synthesis. These data included herein indicates that VNS may treat MS and impact remyelination.

All of the methods and apparatuses described herein, in any combination, are herein contemplated and can be used to achieve the benefits as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates a typical example of the 4 epochs that follow lysolecithin injection to the spinal cord in a model used to study multiple sclerosis.

FIGS. 2A and 2B illustrate the experimental protocols used to study demyelination and remyelination.

FIG. 3A illustrates a cross-section of a healthy spinal cord.

FIG. 3B illustrates a stained cross-section of a spinal cord with a lesion (which may be considered a demyelination) induced by lysolecithin injection.

FIGS. 4A-4D are graphs that show that vagus nerve stimulation reduced the amount of demyelination that resulted from lysolecithin injection. FIG. 4A is a graph showing the effect of vagal nerve stimulation (VNS) on demyelination with various levels of stimulation (O mA, 0.25 mA, and 0.75 mA) four days following inducing of a demyelinating lesion. FIG. 4B shows the increase in remyelination by two weeks after inducing the demyelinating lesion without VNS treatment (0 mA) and with VNS treatment (0.75 mA), showing a rapid remyelination when VNS is applied. FIG. 4C is a 3D graph of the lesion size variation by depth at four days post induction of the demyelinating lesion with and without VNS treatment. FIG. 4D is a 2D projection graph of the median lesion four days post-induction of the demyelinating lesion comparing sham (no VNS treatment) and VNS treatment.

FIGS. 5A-5G are graphs that show that vagus nerve stimulation increased the rate and/or amount of remyelination. FIG. 5A is a graph showing the change in demyelination (determined by the change in induced lesion volume) following induction of demyelination with and without VNS treatment, showing an approximately 65% reduction in the area under the lesion volume (mm³)/days post induction. FIG. 5B is a 3D representation of the demyelination (lesion) size variation with depth for no VNS treatment (sham) and VNS treatment. FIG. 5C is a 2D projection of median lesion volume eight days post-induction of demyelination (e.g., lesion) with VNS treatment and without VNS treatment (sham). FIG. 5D is a 3D representation of demyelination (lesion size) variation with depth at day 14 following inducing of demyelination (day 14 post induction) with VNS treatment (VNS) and without VNS treatment (sham). FIG. 5E is a 2D projection of median demyelination (lesion) at two weeks post-induction of demyelination with VNS treatment and without VNS treatment (“sham”). FIG. 5F is a 3D representation of demyelination (lesion size) variation with depth at day 21 following inducing of demyelination (day 14 post induction) with VNS treatment (VNS) and without VNS treatment (sham). FIG. 5G is a 2D projection of median demyelination (lesion) at three weeks post-induction of demyelination with VNS treatment and without VNS treatment (“sham”).

FIG. 6A shows the experimental protocol used to show the effect of VNS treatment as described herein on vessel leakiness following post-induction of demyelination.

FIG. 6B illustrates the use of VNS treatment as described herein to reduce the leakiness of the blood-brain barrier following induced demyelination. VNS treatment before induced demyelination prevented the passage of dye (Evans blue) through the rat model of the blood brain barrier. VNS treatment after induced demyelination reduced and reversed the leakiness. VNS treatment on Day 0 (following LPC induction) significantly decreased leukocyte infiltration 24 hours post-stimulation, while VNS treatment on Day 4 post-LPC induction significantly decreases leukocyte infiltration 24 hours post-stimulation.

FIG. 7A illustrates the effect of the alpha-7 nicotinic acetylcholine receptors (α7 nAChR) in preventing demyelination re-myelination from VNS treatment (compared to sham without VNS treatment).

FIG. 7B illustrates the effect of the alpha-7 nicotinic acetylcholine receptors in increasing re-myelination from VNS treatment (compared to sham without VNS treatment).

FIG. 8 shows the effect of VNS treatment as described herein to prevent or reverse leakiness of the blood-brain barrier compared to sham (no VNS treatment). In FIG. 9, CD3+ T cell infiltration was significantly decreased in the VNS group on Day 3 post-LPC induction compared to Sham group by 50%.

FIG. 9 illustrates macrophage infiltration through a model of the blood-brain barrier is significantly decreased 24 hours post-demyelination induction (e.g., via LPC) with VNS treatment compared to sham (no VNS treatment) by 55%.

FIGS. 10A and 10B illustrate the effect of pro-resolution lipid Resolvin D1 following induction of demyelination with VNS treatment (VNS) and without VNS treatment (sham), showing Resolvin D1 (RvD1) is increased in VNS animals 4 days post-LPC induction compared to Sham animals and remains elevated 14 days post-LPC induction. Levels were decreased below that of the Sham 21 days post-LPC induction, by which time, no visible lesion is detected in VNS animals.

FIG. 11 schematically illustrates one example of an apparatus for reducing demyelination (e.g., increasing remyelination and/or reducing leakage through the blood-brain barrier), as described herein.

FIG. 12A. shows one example of a prototype vagus nerve stimulator that may be implanted to treat MS as described herein. Implantable devices may include a pulse generator, with a bipolar cuff electrode.

FIG. 12B is a schematic representation of implantation, immunization and VNS treatment. VNS device was implanted on day-7 and rats were immunized with MBP on day 0. Daily VNS treatment began on day 7 and continued through day 21. EAE symptom onset occurred on approximately days 7 and 8.

FIG. 12C shows the effect of VNS treatment vs. Sham and Disease Control, quantified by area under the curve. FIG. 12D is a graph of the maximum clinical score. FIG. 12E shows a graph of the total number of symptomatic days. In FIGS. 12C-F, group means were compared by 2-way mixed model ANOVA (independent variables: treatment, DPSO). Group means were compared by ANOVA followed by Tukey's multiple comparison test. *p<0.05, **p<0.01, ***p<0.001, ****p<0.0001. FIG. 12F shows the change in clinical score over time (days post symptom onset; DPSO). The data is presented as median±IQR and daily group medians were compared with Kruskal-Wallis tests. *p<0.05, **p<0.01.

FIGS. 13A-13B illustrate that VNS and teriflunomide similarly reduce EAE symptoms and duration. FIG. 13A shows a schematic representation of immunization and teriflunomide treatment. Rats were immunized with MBP on day 0. Daily teriflunomide (1 mg/kg or 3 mg/kg) or control (carrier; carboxymethyl cellulose in Tris-HCl pH7.5) treatment (oral gavage) began on day 7 and continued through day 21. EAE symptom onset occurred on approximately days 7 and 8. FIG. 13B shows clinical score over time (days post symptom onset; DPSO). The data is presented as median±IQR. The mean area under the curve between groups were compared by ANOVA followed by Tukey's multiple comparison test. AUC was reduced in the 3 mg/kg/day teriflunomide vs. control (p=0.0269). AUC was not significantly different between 3 mg/kg/day teriflunomide vs. VNS (p=0.5944)

FIG. 13A shows the treatment regime for treating EAE mice. FIG. 13B is a graph showing VNS reduces inflammatory lesion formation and demyelination in EAE spinal cord at peak disease.

FIGS. 13C-13F illustrate that VNS reduces inflammatory lesion formation and demyelination in the EAE SC at peak disease. FIGS. 13C and 13D show 3 μm lumbar SC sections harvested at peak disease that were stained with H&E or LFB with NFR (LFB & NFR) to visualize inflammatory lesions containing cellular infiltrates and WM demyelination, respectively. Representative sections are shown. FIG. 13C shows H&E staining revealed that VNS prevents the formation of inflammatory lesions (arrows) in both WM and GM regions. FIG. 13D shows a reduced loss of myelin was observed in the WM region of VNS vs. Sham rats (arrows). No perceptible inflammatory lesions or loss of myelin was observed in naïve rats.

FIGS. 13E and 13F show quantification of inflammatory lesion and demyelinated area. Data presented as mean±SEM. Group means (n=3 to 4) were compared by ANOVA corrected for multiplicity with Tukey's test. *P<0.05, **P<0.01. Mean Sham lesion area=73,030 μm2. Mean Sham demyelinated area=51,800 μm2. WM, White matter; GM, gray matter.

FIG. 14 is a graph showing EAE disease stages. The EAE stages within which data assessed are shown superimposed over the clinical score over time (data from FIG. 12F).

FIGS. 15A-15D illustrate VNS reduction of inflammatory lesion formation and demyelination in EAE spinal cord at additional EAE stages. 3 μm lumbar spinal cord sections harvested at worsening and remitting EAE stages were stained with hematoxylin and eosin (H&E), shown in FIGS. 15A and 15C, or Luxol fast blue with nuclear fast red (LFB & NFR), shown in FIG. 15B or 15D, to visualize inflammatory lesions containing cellular infiltrates and white matter demyelination, respectively. Representative sections are shown in FIGS. 15A and 15C. VNS prevents the formation of inflammatory lesions in both white matter (WM) and gray matter (GM) regions during worsening and remitting stages. FIG. 15B shows VNS reduced loss of myelin in the WM region vs. Sham rats during worsening and remitting stages.

FIGS. 15E-15I illustrate VNS reduction of microglial/macrophage activation and proliferation, shifting from proinflammatory to proresolving state. FIG. 15E shows representative 40× confocal images of EAE rat lumbar SC sections immunolabeled with microglia/macrophage marker Iba1 colocalized with either the M1 phenotype marker iNOS (proinflammation) or M2 phenotype marker CD206 (proresolution). FIGS. 15F-15I show quantification of Iba1, iNOS, and CD206 reveals significant reduction in both Iba1+ area fraction and percentage of iNOS+ Iba1+ population with concomitant increase in percentage of CD206+ Iba1+ population with VNS as compared to sham. Data presented as mean±SEM. Group means (n=3) were compared by the unpaired t test. **P<0.01, ***P<0.001.

FIGS. 16A-16B show that VNS restricts infiltration of neutrophils into the CNS. FIG. 16A shows representative 20× confocal images of naïve and EAE rat lumbar SC sections immunolabeled with neutrophil marker CD66b during the initial days of symptomatic disease (0-2 DPSO). FIG. 16B shows VNS significantly reduced the CD66b+ cell numbers in lumbar SC sections compared to Sham rats. Data presented as Mean #cells/FOV (0.10 mm2)±SEM. Group means (n=3 or 4) were compared by ANOVA corrected for multiplicity with Tukey's test. **p<0.01.

FIGS. 16C-16F show VNS reduces entry of pathogenic lymphocytes into the CNS during peak EAE. FIGS. 16C and 16D show representative 20× confocal images showing reduced presence of CD4, IL-17, and IFN-γ in VNS SC. 8 μm lumbar SC sections harvested at peak disease were probed with antibodies recognizing CD4, IL-17, IFN-γ, and with DAPI. FIGS. 16E and 16F show lumbar SC cells were digested, isolated, probed with antibodies (against CD45, CD4, IL-17, and IFN-γ), and counted by flow cytometry. VNS reduced CD45+/CD4+/IL17+ and CD45+/CD4+/IFN-γ+ cells. In FIG. 16E the gating strategy with representative results. FIG. 16F CD45+/CD4+/IL17+ and CD45+/CD4+/IFN-γ+ cells reported as a fraction of CD45+ cells and normalized to Sham. Data presented as mean±SEM, for Sham CD45+/CD4+/IL17+ (% CD45+)=33.3±2.7, mean±SEM for Sham CD45+/CD4+/IFN-γ+(% CD45+)=18.5±5.6. n=3 to 6. ns, not significant; ***P<0.001 by the unpaired t test with Welch's correction.

FIGS. 17A-17B shows fibrinogen leakage into the CNS parenchyma. High magnification representative confocal images (63×) showing fibrinogen deposition in CNS parenchyma in SC section (8 μm). Sections were stained with the antibodies for either the vascular endothelial marker CD31 (PCAM1) (FIG. 17A) or tight junction protein claudin-5 (FIG. 17B) and fibrinogen. Image representative of multiple confocal images taken from Sham and VNS rats.

FIGS. 18A-18D show VNS suppresses activation of astrocytes, preserves BBB integrity and restricts fibrinogen deposition at peak EAE. (FIG. 18A) Representative 40× confocal images of naive and EAE rat lumbar SC sections immunolabeled for astrocyte marker GFAP as an index of inflammation. (FIG. 18B) Representative 63× confocal images of lumbar SC sections stained for the inflammatory plasma protein fibrinogen and tight junction protein claudin-5. (FIG. 18C) Quantification of GFAP MFI reveals significant downregulation of GFAP expression with VNS treatment, to comparable levels with naïve animals. (FIG. 18D) VNS significantly reduced fibrinogen deposition and maintained claudin-5 structural integrity in the CNS. Data presented as the integrated density, normalized to Sham, of individual focal point integrations, as well as mean±SEM. Group means (n=3) were compared by ANOVA corrected for multiplicity with Tukey's test, *P<0.05, **P<0.01, ***P<0.001.

FIGS. 19A-19D show that VNS modulates gene expression pathways at 0 to 2 DPSO. RNA was purified from lumbar SC at 0 to 2 DPSO and quantitative PCR to genes of interest was performed. FIG. 19A shows VNS modulates Th1 and Th17-related inflammatory gene expression, Ifng, Il12, and Il17. FIG. 19B shows VNS modulates gene expression of key inflammatory reflex components Adrb2, Chat, and Charna7. FIG. 19C shows VNS up-regulates expression of Mbp, Mog, Plp, genes involved in myelin synthesis and oligodendrocyte differentiation and FIG. 19D shows Mbp gene transcription factors Sp1, Sox10, and Pur alpha. In FIGS. 19A-19D, VNS-mediated gene expression levels were calculated as relative fold change over the Actin beta control gene and normalized to Sham (dotted line). Data presented as mean±SEM. Change from Sham values was analyzed by the two-tailed unpaired t test with Welch's correction. *P<0.05, **P<0.01, ***P<0.001.

FIG. 20 is a table (table 2) showing clinical severity over time in control, sham and VNS.

FIG. 21 is a table (Table 3) showing weight over time in control, sham and VNS.

### DETAILED DESCRIPTION

Electrical and/or mechanical stimulation of the cholinergic anti-inflammatory pathway (NCAP) by stimulation of the carotid vagus nerve have been well described. For example, see U.S. Pat. Nos. 6,838,471, 8,914,114, 9,211,409, 6,610,713, 8,412,338, 8,996,116, 8,612,002, 9,162,064, 8,855,767, 8,886,339, 9,174,041, 8,788,034 and 9,211,410, each of which is herein incorporated by reference in its entirety. It has not previously been suggested that vagus nerve stimulation may be used to prevent or reduce demyelination and/or improve remyelination. Vagus nerve stimulation, through activation of both efferent and afferent pathways (or primarily through one of the efferent or afferent pathway), may be able to reduce the inflammation associated with inflammatory diseases and disorders, thereby reducing the severity of the symptoms and/or slowing, stopping, or reversing the progression of the disease. Applicants have surprisingly found that the apparatuses (e.g., systems, devices, etc.) and methods described herein may be used to stimulate the vagus nerve to reduce demyelination and/or to increase or promote remyelination. Furthermore, although the use of VNS treatment to modulate inflammation has been thought to involve afferent pathways, remyelination and demyelination may involve the efferent pathway or both the afferent and efferent pathways.

Diseases (e.g., diseases and disorder of myelination) which may benefit from VNS include, but are not limited to, multiple sclerosis (MS), Alzheimer's disease, Parkinson's disease, Huntington's disease, Amyotrophic lateral sclerosis (ALS), chronic inflammatory demyelinating polyneuropathy (CIDP), and Batten disease. Neuropathies that may benefit from VNS include peripheral neuropathies, cranial neuropathies, and autonomic neuropathies.

### Vagus Nerve Stimulation Systems and Devices

In some variations the devices described herein are electrical stimulation devices that may be implanted and may be activated to apply current for a proscribed duration, followed by a period without stimulation. As described in the examples that follow, the stimulation protocol may comprise a very limited period of stimulation (e.g., an on-time of less than 5 minutes, 2 minutes, 1 minute, etc.) followed by an off-time (during which stimulation is not applied, and may be prevented from being applied) of extensive duration (e.g., greater than 10 minutes, 20 minutes, 30 minutes, 40 minutes, 50 minutes, 1 hour, 1.5 hours, 2 hours, 4 hours, 12 hours, greater than 20 hours, greater than 24 hours, greater than 36 hours, greater than 48 hours, etc.). The applied energy may be electrical energy that is a fixed current having a frequency that is within the range of about 0.5 mA to 5 mA (e.g., approximately 2 mA), at a frequency of between about 1 Hz and about 1000 Hz (e.g., between 1 Hz and 100 Hz, between 1 Hz and 30 Hz, between 10 Hz and 200 Hz, etc.), where the pulses applied have a pulse width of approximately (50-500 usec, e.g., a 200 usec pulse). Thus, the duty-cycle of the applied current may be extremely low, where duty cycle may refer to the ratio of on-time/(on-time plus off-time). The stimulation is applied at an extremely low duty cycle, where duty cycle may refer to the percent of on-time to the total on-time and off-time for the ongoing treatment. For example, low duty cycle may be less than about 10, 5, 4, 3, 2, 1, or 0.5 percent of on-time to the total on time and off-time. The effect may be seen relatively quickly and may persist over the entire off-time.

In particular, the methods and apparatuses described herein may be applied as needed, e.g., when the patient expresses or is likely to express an increased risk for demyelination and/or is experiencing (or has experienced) demyelination. Alternatively or additionally, the methods an apparatuses may be applied as needed when the patient expresses or is likely to express, and/or is experiencing (or has experienced) a leakage through the blood-brain barrier.

For example, we show herein that a low level, low duty cycle stimulation protocol (as described herein) reduces demyelination and/or increases remyelination, and prevents and/or reduces leakage through the blood-brain barrier. The effectiveness of low level, low duty cycle vagus nerve stimulation (VNS therapy) administered on even a single day results in a reduction in demyelination and an increase in remyelination seen over the course of two to three weeks. This type of stimulation contrasts with the use of a high duty cycle stimulation used by others to modulate vagus-nerve mediated functions (such as heart rate, etc.), or treat disorders such as epilepsy and depression. An important finding here is that demyelination can be reduced and even more surprisingly, remyelination can be increased. This effect is corroborated at these low duty cycle parameters by examining the histology of the spinal cord as described later below. Although low duty cycle vagus nerve stimulation is effective and highly efficient at reducing inflammation, in some embodiments, a higher duty cycle stimulation can be used, such as a duty cycle that is greater than about 1, 2, 3, 4, 5, 10, 20, 30, 40, or 50 percent of on-time to the total on-time and off-time.

MS patients may experience circadian pattern disruptions to symptoms that may be associated with or caused in part by the circadian patterns of IL-6 levels. Optionally, drugs, such as steroids, can be used along with VNS to suppress nighttime spiking of IL-6. Similarly, VNS can be modulated, by altering the timing of the stimulations for example, to suppress nighttime spiking of IL-6 more effectively. However, one advantage of VNS is the relatively long duration of the effect after a single stimulation, which may allow suppression of IL-6 levels during both night and day, which may render unnecessary the need for supplementary drug treatment or alternative timings. In some embodiments, VNS can be given in the evening before sleep, such as 15, 30, 45, 60, 90, 120, 150, or 180 minutes before sleep, and may also be given at night during sleep, to ensure nighttime suppression of IL-6 levels. In some embodiments, the amplitude of stimulation during sleep can be lowered (e.g., less than 2, 1.5, or 1 mA) to avoid waking the patient. In some embodiments, IL-6 levels can be measured and/or monitored, and VNS can be modulated based on the measured and/or monitored IL-6 levels. Other cytokines may also be measured and/or monitored, such as IL-1, TNF, IFN-gamma, IL-12, IL-18, and GM-CSF. These other cytokines may be used instead of or in addition to IL-6, either in combination or singly.

The methods, devices and systems herein may be applied specifically to treat any disorder for which a reduction of demyelination and/or an increase in remyelination would be beneficial. For example, described herein are electrodes (e.g., cuff electrodes, microstimulators) that may be placed around the vagus nerve and may communicate with one or more stimulators configured to apply appropriate stimulation of the vagus nerve to modulate demyelination and/or remyelination. The stimulator may be implanted. In some variations the stimulator is integral to the electrodes and may be charged externally. The extremely low duty-cycle of the technique described herein may allow the device to be miniaturized to a greater degree than previously suspected for the treatment of chronic disorders via an implantable device.

In general, a device or system for modulating demyelination and/or remyelination may include a stimulator element (e.g., an electrode, actuator, etc.) and a controller for controlling the application of stimulation by the stimulator element. A stimulator element may be configured for electrical stimulation (e.g., an electrode such as a cuff electrode, needle electrode, paddle electrode, non-contact electrode, array or plurality of electrodes, etc.), mechanical stimulation (e.g., a mechanical actuator, such as a piezoelectric actuator or the like), ultrasonic actuator, thermal actuator, or the like. In some variations the systems and/or devices are implantable. In some variations the systems and/or device are non-invasive. In general, the controller may include control logic (hardware, software, firmware, or the like) to control the activation and/or intensity of the stimulator element. The controller may control the timing (e.g., on-time, off-time, stimulation duration, stimulation frequency, etc.). In variations in which the applied energy is electrical, the controller may control the applied waveform (amplitude, frequency, burst duration/inter-burst duration, etc.). Other components may also be included as part of any of these device or system, such as a power supply (e.g., battery, inductive, capacitor, etc.), transmit/receive elements (e.g., antenna, encoder/decoder, etc.), signal generator (e.g., for conditioning or forming the applied signal waveform), and the like. In some embodiments, a rechargeable battery that may be inductively charged allows the stimulator to deliver numerous electrical stimulations before needing to be recharged. In other embodiments, one or more capacitors that can also be inductively charged can be used to store a limited amount of energy that may be sufficient to deliver a single stimulation or a daily amount of stimulations. This dramatically reduces the size and cost of the stimulator but requires that the user charge the stimulator daily or before each use.

In one example, an implantable device for modulating demyelination and/or remyelination (and/or reducing or preventing leaking of the blood-brain barrier) includes an electrode for electrically stimulating the vagus nerve. The electrode may be, for example, a cuff electrode. The electrode may be connected (directly or via a connector) to a controller and signal generator. The signal generator may be configured to provide an electrical signal to the electrode(s). For example, the electrical signal may be an electrical waveform having a frequency of between about 0.1 Hz and about 1 KHz (e.g., 10 Hz), where the pulses applied have a pulse width of approximately (50-500 usec, e.g., a 200 usec pulse). The signal generator may be battery (and/or inductively) powered, and the electrical signal may be amplitude and/or voltage controlled. For example in some variations the device or system may be configured to apply a current that is between about 0.05 mA to 25 mA (e.g., approximately 0.5 mA, 1 mA, 2 mA, 3 mA, etc.). The electrical signal may be sinusoidal, square, random, or the like, and may be charge balanced. In general, the controller (which may be embodied in a microcontroller such as a programed ASIC), may regulate turning on and off the stimulation. For example, stimulation may be applied for an on-time of between about 0.1 sec and 10 minutes (e.g., between 1 sec and 5 minutes, between 1 sec and 2 minutes, approximately 1 minute, etc.); the stimulation may be configured to repeat automatically once every x hours or days, e.g., every other day (off time of approximately 48 hours), once a day (e.g., with an off-time of approximately 24 hours), twice a day (off-time of approximately 12 hours), three times a day (off time of approximately 8 hours), four times a day (off time of approximately 6 hours), or the like. In some variations the implant may be configured to receive control information from a communications device. The communications device may allow modification of the stimulation parameters (including off-time, on-time, waveform characteristics, etc.). The communications device may be worn, such as a collar around the neck, or handheld.

In use, an implant may be configured to be implanted so that the electrodes contact or approximate the vagus nerve or a portion of the vagus nerve. In one variation the implant includes a cuff that at least partially surrounds the vagus (e.g., near the carotid region). The controller and/or signal generator (including any power source) may be formed as part of the cuff or may be connected to by a connector (e.g., wire).

In some variations the device may be non-invasive. For example, the device may be worn outside the body and may trigger stimulation of the vagus nerve from a site external to the body (e.g., the car, neck, torso, etc.). A non-invasive device may include a mechanical device (e.g., configured to apply vibratory energy). In some variations the device is configured to apply ultrasound that may specifically target the vagus nerve and apply energy to activate the vagus nerve. In some variations, transcutaneous magnetic stimulation of the vagus nerve may be used.

In any of the variations described herein, the devices, system and methods may be configured to prevent desensitization of the signal in a way that would reduce or inhibit the modulation of demyelination and/or remyelination. For example in some variations, “over stimulation” of the vagus nerve, e.g., simulation at intensities that are too great or applied for too long, or outside of the frequency ranges described herein, may result in desensitization of the effect, thus further modulation may be limited or inhibited. Therefore, in some embodiments, the amplitude of stimulation may be restricted from exceeding (i.e., be less than) about 3 mA, 4 mA, or 5 mA, and/or the duty cycle may be restricted from exceeding about 1, 2, 3, 4, 5, 6, 7, 8, 9, 10, 15, 20, or 25%. In some embodiments, the amplitude is also at least 0.25 mA, 0.5 mA, 0.75 mA, or 1.0 mA.

The examples illustrated above may provide insight into the devices, systems and methods of use for stimulation of the vagus nerve to modulate demyelination and/or remyelination. These methods and devices may be used to treat any indication for which modulation of demyelination and/or remyelination would be beneficial. Non-limiting examples of indications include neurodegenerative and neuroinflammatory diseases such as multiple sclerosis (MS), Alzheimer's disease, Parkinson's disease, Huntington's disease, Amyotrophic lateral sclerosis (ALS), and Batten disease. Other examples include peripheral neuropathies, cranial neuropathies, and autonomic neuropathies. In general, these devices may offer alternative and in some ways superior treatment as compared to pharmacological interventions aimed at modulating demyelination and/or remyelination and therefore may be used for any indication for which such pharmacological treatments are suggested or indicated. In some embodiments, the VNS treatments described herein can be used in conjunction with pharmacological treatments, particularly when the pharmacological treatment has a different mechanism of action than the VNS, which may lead to synergistic results.

Thus, the methods of modulating demyelination and/or remyelination as described herein may be used in conjunction with one or more pharmacological interventions, and particularly interventions that treat diseases associated with demyelination, neurodegeneration or neuroinflammation. For example, it may be beneficial to treat a subject receiving stimulation of the vagus nerve to modulate demyelination and/or remyelination by also providing agent such as intravenous corticosteroids (e.g., methylprednisolone), oral corticosteroids, interferons beta-la and beta-1b, monoclonal antibodies (e.g., natalizumab, alemtuzumab, daclizumab and ocrelizumab), and immunomodulators (e.g., glatiramer acetate, mitoxantrone, fingolimod, teriflunomide, and dimethyl fumarate).

Thus, described herein are devices (VNS devices) for the treatment of neurodegenerative and/or neuroinflammatory disorders. Such devices are generally configured to apply low duty-cycle stimulation to the vagus nerve of a subject, as described in any of the variations (or sub-combinations) of these variations. In some embodiments, the patient is first diagnosed or identified with a neurodegenerative or neuroinflammatory disorder, particular a disorder characterized by demyelination or need for remyelination, before being implanted and treated with the VNS device.

In use, any of the methods described herein may include a step of monitoring for demyelination or demyelination-associated disorders, which may be determined through detection of a biomarker from blood and/or cerebrospinal fluid, and/or through medical imaging techniques such as MRI or CT scans. For example, as assay for an inflammatory cytokine (e.g., tumor necrosis factor) may be used to detect acute inflammatory episodes. Monitoring may be continuous or discrete (e.g., at one or more times, or time intervals). In addition or alternatively, biomarkers associated with multiple sclerosis or other neurodegenerative or neuroinflammatory diseases or neuropathies can be used for monitoring, depending on the disease being treated. See Housley, W. J., D. Pitt and D. A. Hafler (2015). “Biomarkers in multiple sclerosis.” Clin Immunol 161 (1): 51-58; and Katsavos, S. and M. Anagnostouli (2013). “Biomarkers in Multiple Sclerosis: An Up-to-Date Overview.” Mult Scler Int 2013:340508. For example, biomarkers found in MS serum and cerebrospinal fluid include markers of neurodegeneration including neurofilament and GFAP, the monocyte macrophage marker CD163, the glial activation marker YKL-40, the B cell chemoattractant CXCL13, miRNA and mRNA, myelin reactive t cells, Kir4.1 antibodies, osteopontin, and microbiome associated lipopeptides. Any of these biomarkers can be monitored and/or measured alone or in combination and can be used as feedback to modulate VNS. Other biomarkers for treating MS patients in particular are listed in Table 1.

TABLE 1
Biomarkers in Multiple Sclerosis
(A) Diagnostic biomarkers (criteria i, iv, v, and vi)

| | | |
|---|---|---|
| (1) Genetic-immunogenetic | | |
| HLA-DRB1*1501 | +++ Risk for MS | See also |
| | | B, E |
| DR3 and DR4 haplotypes | ++ Risk for MS | |
| HLA-DRB1*04 | ++ Risk for MS | |
| HLA-DRB1*0401 | + Risk for high familial autoimmunity in | See also |
| | MS patients | F |
| HLA-DQ1*0102 | + Risk for MS, in coexistence with HLA- | |
| | DRB1*1501 | |
| HLA-DPB1*0501 | + Risk for opticospinal MS | |
| HLA-DPB1*0301 | + Risk for opticospinal MS | |
| IL2RA and IL7RA | + Risk for MS | |
| polymorphisms | | |
| EVI5, CD58, KIAA0350, and | +/− Risk for MS | |
| RPL5 polymorphisms | | |
| (2) Laboratorial | | |
| OCB IgG | +++ But with low specificity | See also |
| | | E |
| KFLC | +++ But with low specificity | See also |
| | | E |
| MRZ reaction | +++ Higher specificity than OCB IgG | See also |
| | | E, F |
| Anti BRRF2, anti EBNA-1 | ++ | See also |
| | | B, C |
| Anti MBP 48-70 and 85-170 | + | See also |
| | | B, E |
| Anti MBP 43-68 and 146-170 | + Differential diagnosis with OND's | See also |
| | | B, E |
| MBP/MOG conformational | + But low specificity | See also |
| epitopes antibodies | | B, E, F |
| VEGF-A | + Lower CSF levels in all disease forms, | See also |
| | but low specificity | D, E |
| Vitamin D | +++ Lower levels, higher risk for MS | See also |
| | | C, F |
| TRECs | + Lower serum levels in all disease forms, | See also |
| | but low specificity | B |
| CSF levels of lipocalin 2 | + Higher CSF levels in MS, but low | See also |
| | specificity | F |
| AR | +++ Differential diagnosis of MS and | See also |
| | NMO | C, E |
| NO and NO metabolites | + Higher CSF and serum levels in MS, but | See also |
| | low specificity | C, E |
| NF-L | ++ Higher CSF levels in MS patients | See also |
| | | C, F |
| NAA | +++ Differential diagnosis of RRMS and | See also |
| | NMO | D, E |
| GFAP | +++ Differential diagnosis of MS and | See also |
| | NMO | C, E |
| | + Differential diagnosis of MS and NMO | See also |
| | | C, E |
| Nogo-A | ++ For MS forms with prominent | See also |
| | neurodegenerative element | D |
| (3) Imaging | | |
| Contrast-enhanced T1 lesions | +++ | See also |
| | | C |
| Hyperintense T2-weighted lesions | +++ | See also |
| | | C, D, E |
| Corpus callosum DTI | ++ Early diagnostic biomarker | See also |
| abnormalities | | E |
| MRS findings (glutamate/choline) | +++ | See also |
| | | C, D, E |
| PET | ++ But still experimental | |
| EPs | +++ | See also |
| Motor EPs | +++ Spinal cord syndrome at presentation | C, |
| VEMPs | +++ Brainstem dysfunction | D, E |
| SSR | ++ Autonomic dysfunction assessment in | See also |
| | MS patients | E |

(B) Biomarkers of phenotypical expression (criteria ii, iv, v, and vi)

| | | |
|---|---|---|
| (1) Genetic-immunogenetic | | |
| HLA-DRB1*1501 | +++ Early disease onset | See also |
| | | A, E |
| HLA-DRB1*1501 | + Risk for cognitive decline | |
| HLA-DRB1*01 | ++ Protection against malignant disease | |
| | form | |
| ApoE ε4 | ++ Greater risk for mental disorders | |
| (2) Laboratorial | | |
| OCB IgM against myelin lipids | +/− Aggressive disease course | See also |
| | | E |
| EBV antibodies | + Early disease onset | See also |
| | | A, C |
| Anti-MBP | +++ ADEM-like onset in childhood MS | See also |
| | | A, E |
| Anti-MOG | +++ Childhood MS, ADEM, isolated optic | See also |
| | neuritis, anti-AQP4 (−) NMO | A, E, F |
| rMOG index | +++ Progressive disease forms | |
| IL-6 serum levels | +++ Age at onset | See also |
| | | C |
| TRECs | ++ Lower levels PPMS | See also |
| | | A |
| Amyloid- (1-42) | ++ Lower levels, higher risk for mental | |
| | disorders | |
| (3) Imaging | | |
| UCCA atrophy | +++ Progressive disease forms | See also |
| | | E |
| NAGM DTI abnormalities | +++ Progressive disease forms | |

(C) Biomarkers of demyelination-neuroinflammation-relapse (criteria i, ii, iii, iv, v, and vi)

| | | |
|---|---|---|
| (1) Genetic-immunogenetic | | |
| TOB1 | +++ Underexpression, higher Th1 and | See also |
| | Th17 percentage | E |
| (2) Laboratorial | | |
| EBV antibodies | + Higher inflammatory activity | See also |
| | | A, B |
| CXCL13 | ++ Mobilizes B-cells, T-helper cells | |
| CXCL12 | +/− Neuroprotection against inflammation | |
| | in EAE/experimental | |
| IFN-/TNF-a | +++ Th1 immune response | |
| IL-1 levels imbalance | + Triggering factor for neuroinflammation | |
| IL-6 | +++ B-cell and T-cell immunity link, Th17 | See also |
| | immune response triggering factor | B |
| | ++ Correlation with relapse frequency in | |
| | female MS patients | |
| IL-10 -592 position | ++ Regulation of CNS autoimmunity | |
| polymorphisms | | |
| IL-15 | ++ BBB disruption, enhanced CD8(+) T | |
| | cytotoxicity | |
| IL-33 | + Increase in IFN-γ and IL-17 in mice EAE | |
| sICAM-1 | ++ Higher levels, higher inflammatory | See also |
| | activity | F |
| | +++ Higher levels in NMO than MS- | |
| | marker of BBB disruption | |
| sVCAM-1 | +++ Higher levels in NMO than MS- | See also |
| | marker of BBB disruption | F |
| Laminin 411 | ++ TH-17 enhancement | |
| 4 Integrin | ++ Correlation with gadolinium-enhanced | See also |
| | lesions during CIS | E, F |
| Osteopontin | ++ Serum and CSF elevation during | |
| | relapse | |
| Fetuin-A | +++ Overexpression in active | See also |
| | demyelinating lesions | F |
| Vitamin D | +++ High levels, anti-inflammatory role- | See also |
| | lower radiological disease activity | A, F |
| CSF mature B-cells/plasma-blasts | ++ Bigger accumulation, higher | |
| | inflammatory activity | |
| CXCR3 | ++ Helps T-cells to enter the brain | |
| CX(3)CR1 | ++ CD4(+)CD28(−) cytotoxic cells | |
| | biomarker | |
| CSF CCR2(+)CCR5(+) T cells | +++ Increase during MS relapse- | |
| | osteopontin enhancement | |
| CD56 Bright NK | ++ Remission phase | |
| AR | +++ Biomarker of BBB disruption | See also |
| | | A, E |
| MMP-9 | ++ Higher CSF levels during relapse | |
| Ninjurin-1 | ++ Upregulation in active demyelinating | |
| | lesions | |
| MBP and fragments | +++ Higher CSF levels during relapse | See also |
| | | F |
| B-Crystalline | +++ Over-expression in active | |
| | demyelinating lesions | |
| NO and metabolites | ++ | See also |
| | | A, E |
| 7-Ketocholesterol | ++ | |
| Glutamate | +++ Higher levels in active demyelinating | |
| | lesions | |
| Cystine/glutamate antiporter | + Over-expression in active demyelinating | |
| | lesions | |
| NF-L | +++ Higher CSF levels, especially the 3rd | See also |
| | week after relapse onset | A, F |
| GFAP | ++ Higher levels during relapse | See also |
| | | A, E |
| S100B | +/− Higher CSF levels during MS/NMO | See also |
| | relapse | A, E |
| N-CAM | + CSF elevation at remission onset | |
| BDNF | ++ Lower levels inhibit demyelination and | See also |
| | axonal loss | D, E, F |
| (3) Imaging | | |
| Contrast-enhanced T1 lesions | +++ Active lesions | See also |
| | | A |
| Hyperintense T2-weighted lesions | ++ Combination of different mechanisms | See also |
| | | A, D, E |
| MTR decrease | + Demyelination and axonal loss combined | See also |
| | | D |
| DTI abnormalities | ++ Combination of different mechanisms | See also |
| | | D, E |
| MRS findings (especially changes | +++ Active lesions | See also |
| in glutamate and choline) | | A, D, E |
| DTS | ++ Promising but still experimental | See also |
| | | D |
| EP's delayed conduction | ++ Demyelination biomarker | See also |
| | | A, D, E |

(D) Biomarkers of axonal loss-neurodegeneration (criteria i, iv, v, and vi)

| | | |
|---|---|---|
| (1) Laboratorial | | |
| VEGF-A | ++ Lower levels, higher risk for | See also |
| | neurodegeneration | A, E |
| 14-3-3 | +/− Axonal loss | |
| NAA | +++ Axonal loss | See also |
| | | A, E |
| BDNF | ++ Lower levels inhibit demyelination and | See also |
| | axonal loss | C, E, F |
| Nogo-A | +++ Higher CSF levels, failure in axonal | See also |
| | repair | A |
| (2) Imaging | | |
| RNFL thinning | +++ Axonal loss in the optic nerve | See also |
| | | E, F |
| Hyperintense T2-weighted lesions | ++ Combination of different mechanisms | See also |
| | | A, C, E |
| Black holes | +++ Axonal loss | See also |
| | | E |
| MTR decrease | ++ Demyelination and axonal loss | See also |
| | combined | C |
| DTI abnormalities | ++ Combination of different mechanisms | See also |
| | | C, E |
| MRS findings (especially NAA) | ++ | See also |
| | | A, C, E |
| DTS | +++ Promising but still not widely | See also |
| | accessible | C |
| Visual and motor EPs | ++ | See also |
| | | A, C, D |

(E) Prognostic biomarkers-biomarkers of disability progression (criteria ii, iv, v, vi, and viii)

| | | |
|---|---|---|
| (1) Genetic-immunogenetic | | |
| HLA-DRB1*1501 | +/− Early progression from RRMS to | See also |
| | SPMS | A, B |
| HLA-DRB1*1501 | + Worst brain atrophy measures | |
| HLA-DQB1*0301 | + Worst brain atrophy measures | |
| HLA-DQB1*0602 | + Worst whole and gray matter atrophy | |
| | measures | |
| TOB1 | +++ Early conversion from CIS to CDMS | See also |
| | | C |
| (2) Laboratorial | | |
| OCB IgG | +++ Conversion from CIS to CDMS | See also |
| | | A |
| KFLC | +++ Conversion from CIS to CDMS | See also |
| | | A |
| OCB IgM | +/− Bad prognostic biomarker | See also |
| | | B |
| MRZ reaction | +++ Conversion from CIS to CDMS | See also |
| | | A, F |
| Anti-MBP | +/− Conversion from CIS to CDMS | See also |
| | | A, B |
| Anti-MOG | +/− Conversion from CIS to CDMS | See also |
| | | A, B, F |
| AR | ++ Marker of clinical severity in NMO | See also |
| | | A, C |
| VEGF-A | ++ Lower levels, progression from RRMS | See also |
| | to SPMS | A, D |
| NO and NO metabolites | ++ Higher CSF levels, longer | See also |
| | relapses/higher disability progression rates | A, C |
| NF-H | +++ Higher CSF levels, progressive | |
| | forms/bad prognostic biomarker | |
| NF-H and tau | +++ Combined high CSF levels, | |
| | conversion from CIS to CDMS | |
| Tubulin/actin | ++ Higher CSF levels, progressive | |
| | forms/worst disability scores | |
| NAA | +++ Lower CSF levels, progressive | See also |
| | forms/worst disability scores | A, D |
| GFAP | ++ Higher CSF levels, progressive MS | See also |
| | forms/worst disability scores | A, C |
| | +++ Disability progression in NMO | |
| S100B | + Disability progression in NMO | See also |
| | | A, C |
| BDNF | ++ Lower CSF levels in SPMS patients | See also |
| | | C, D, F |
| Unblocked α4 integrin | + Prognostic factor of risk for PML | See also |
| | | C, F |
| (3) Imaging | | |
| RNFL thinning | + Correlation with brain atrophy measures | See also |
| | and disease progression | D, F |
| Hyperintense T2-weighted lesions | +/− | See also |
| | | A, C, D |
| Black holes | +/− | See also |
| | | D |
| Whole brain atrophy measures | ++ Worsening rates at MS onset, | |
| | prognostic biomarker of disability after 8 | |
| | years | |
| Gray matter atrophy measures | +++ Higher worsening rates, progressive | |
| | forms/early CIS conversion to RRMS | |
| UCCA atrophy | ++ Progressive forms, good correlation | See also |
| | with EDSS, bad prognostic in RRMS | B |
| DTI abnormalities | +++ Early prognostic biomarker of relapse | See also |
| | | C, D |
| Corpus callosum DTI | +++ Bad prognostic biomarker | See also |
| abnormalities | | A |
| Spinal cord DTI abnormalities | +++ Good correlation with EDSS scores | |
| Early MRS abnormalities | ++ Bad prognostic biomarker | See also |
| | | A, C, D |
| Combined EPs | +++ Good prognostic biomarker, especially | See also |
| | for benign disease forms | A, C, D |
| SSR | ++ Correlation with higher EDSS scores | See also |
| | | A |

(F) Biomarkers of therapeutical response (criteria i, iv, v, vi, and vii)

| | | |
|---|---|---|
| (1) Genetic-immunogenetic | | |
| HLA-DRB1*0401, 0408, 1601 | +++ Higher risk for developing | See also |
| | neutralizing antibodies against IFN-B | A |
| (2) Laboratorial | | |
| MRZ reaction | ++ B-cell immunity targeted therapy | See also |
| | | A, E |
| Anti-MOG | ++ B-cell immunity targeted therapy | See also |
| | | A, B, E |
| Fetuin-A | +++ Decreased CSF levels in Natalizumab | See also |
| | responders | C |
| MBP | +++ Decrease in CSF levels in | See also |
| | methylprednizolone responders | C |
| CSF lipocalin 2 | ++ Decreased CSF levels in Natalizumab | See also |
| | responders | A |
| Unblocked α4 integrin | +++ Therapeutical response to | See also |
| | Natalizumab | C, E |
| NF-L | +++ Normalized CSF levels in | See also |
| | Natalizumab responders | A, C |
| BDNF | +++ CSF elevation in Glatiramer Acetate | See also |
| | responders | C, D, E |
| TRAIL | ++ Serum levels good predictors of | |
| | response in IFN-B | |
| MxA | ++ Serum levels good predictors of | |
| | response in IFN-B | |
| sVCAM | ++ CSF alterations in IFN-B responders | See also |
| | | C |
| Th17 immune profil | +/− Immune response exacerbation by | |
| | IFN-B | |
| Vitamin D | +++ Increased levels in IFN-B responders | See also |
| | | A, C |
| sICAM-1 | + Lower levels in Cladribine responders | See also |
| | | C |
| sE-Selectin | + Lower levels in Cladribine responders | |
| (3) Imaging | | |
| RNFL | +++ Biomarker of therapeutical efficacy | See also |
| | for several agents | D, E |

Classification of biomarkers.
+++ very strong correlation, ++ strong correlation, + modest correlation, and +/− controversial correlation.
Criteria used for classification., (i) Biological rationale; (ii) clinical rationale; (iii) predictability of disease initiation, reactivation or progression, or of disease differentiation; (iv) sensitivity and specificity; (v) reproducibility; (vi) practicality; (vii) correlation with therapeutical outcome; (viii) correlation with prognosis and disability.
Biomarkers of more than one category are indicated in the third column.

The information described herein for the first time shows that stimulation of the vagus nerve modulates demyelination and/or remyelination and/or leaking through the blood-brain barrier. The examples provided herein are not intended to be comprehensive but merely illustrate and embody certain variations of the invention. It is within the abilities of one of ordinary skill in the art to understand and apply, without undue experimentation, the invention as described herein.

### Example 1

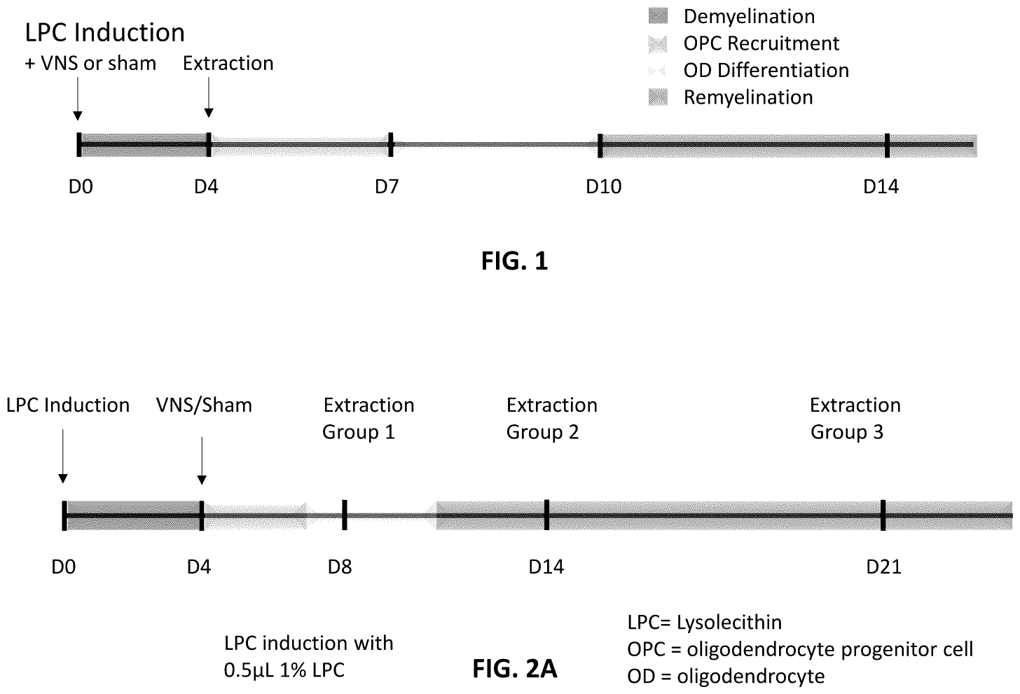

To study the effect of VNS on neurodegeneration and neuroinflammation, a lysolecithin (LPC)-induced MS model can be used. Lysolecithin is a bioactive pro-inflammatory lipid that is a detergent-like membrane solubilizing agent. A 1% solution of LPC can induce local demyelinating lesions when injected into the white matter of the spinal cord. Four distinct epochs occur over 14 days post-injection: (1) demyelination; (2) oligodendrocyte progenitor cell (OPC) recruitment; (3) differentiation; and (4) remyelination. FIG. 1 illustrates a typical example of the 4 epochs, where demyelination occurs from about days 0-3, OPC recruitment occurs from about days 3-7, OPC differentiation occurs from about days 7-10, and remyelination occurs from about days 10-14.

To induce a self-limited demyelinating lesion, spinal cords of female BALB/c mice were injected between T3-T5 with 1% LPC (0.5 μL at 0.25 μL/min). The procedure to inject the mice with LPC was as follows. The mouse was anesthetized and stabilized into a stereotaxic frame. A midline incision was made between the scapulae. The underlying fat pads were bluntly separated and the spinous process of the T2 vertebra was identified, and a laminectomy was performed. A syringe was advanced to 0.3 mm into the spinal cord and 0.5 μL of LPC was injected at a rate of 0.250 μL/min for 2 min. The muscle and adipose tissue were sutured, and the skin was closed with surgical staples

VNS was performed as previously described (Olofsson, Levine, et al. 2015. Bioelectronic Medicine: 37-42) on Day 0 or Day 4 post-induction with LPC. More specifically, to study the effect of VNS on demyelination, VNS (0.75-1 mA, 250 μS pulse, 10 Hz) or sham VNS (0 mA) was performed immediately following LPC administration, and the mice were euthanized on the day of expected peak lesion volume (day 4 post-induction; J Neurocytol 24(10): 775-81). The demyelination experimental protocol is summarized in FIG. 2A.

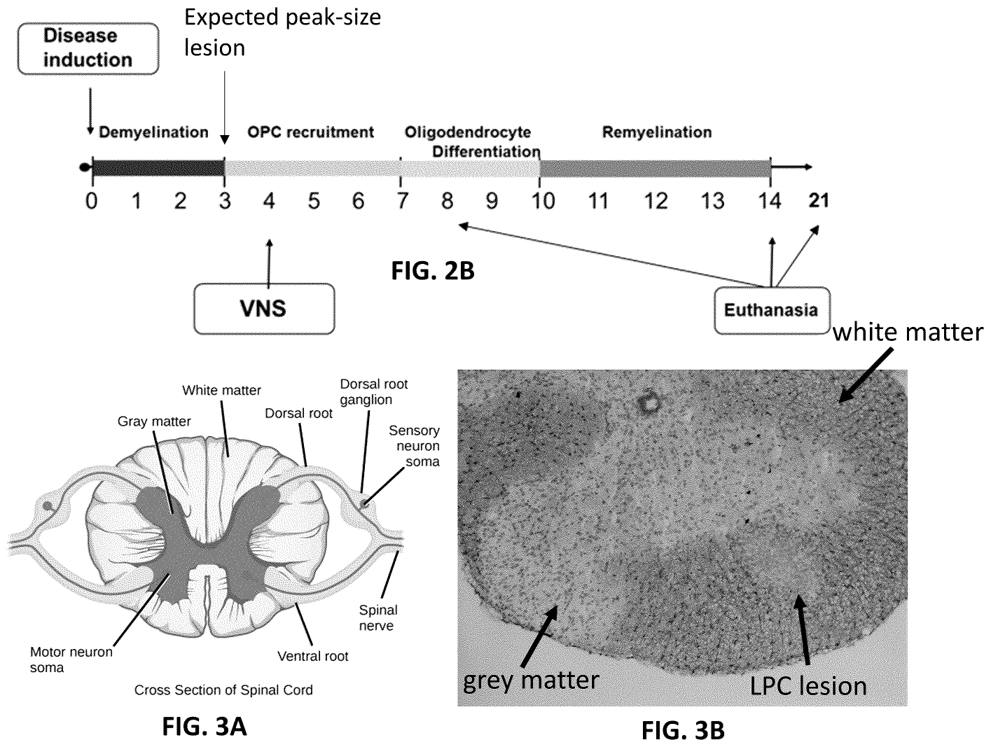

Spinal cord lesion volumes/areas were quantified by myelin loss as assessed from luxol blue-stained, 15 μm serial sections. FIG. 3A shows an illustration of a typical cross-section of the spinal cord, and FIG. 3B shows a luxol blue stained cross-section of the spinal cord with an LPC induced lesion in the anterior funiculus of the white matter 5 days post-LPC injection. To study the effect of VNS on remyelination, VNS or sham VNS treatments was performed 4 days post-induction, mice were euthanized on days 8, 14, or 21 post-induction, and nerves were processed as above. The remyelination experimental protocol is summarized in FIG. 2B. Mean lesion volumes between groups were compared by t-test.

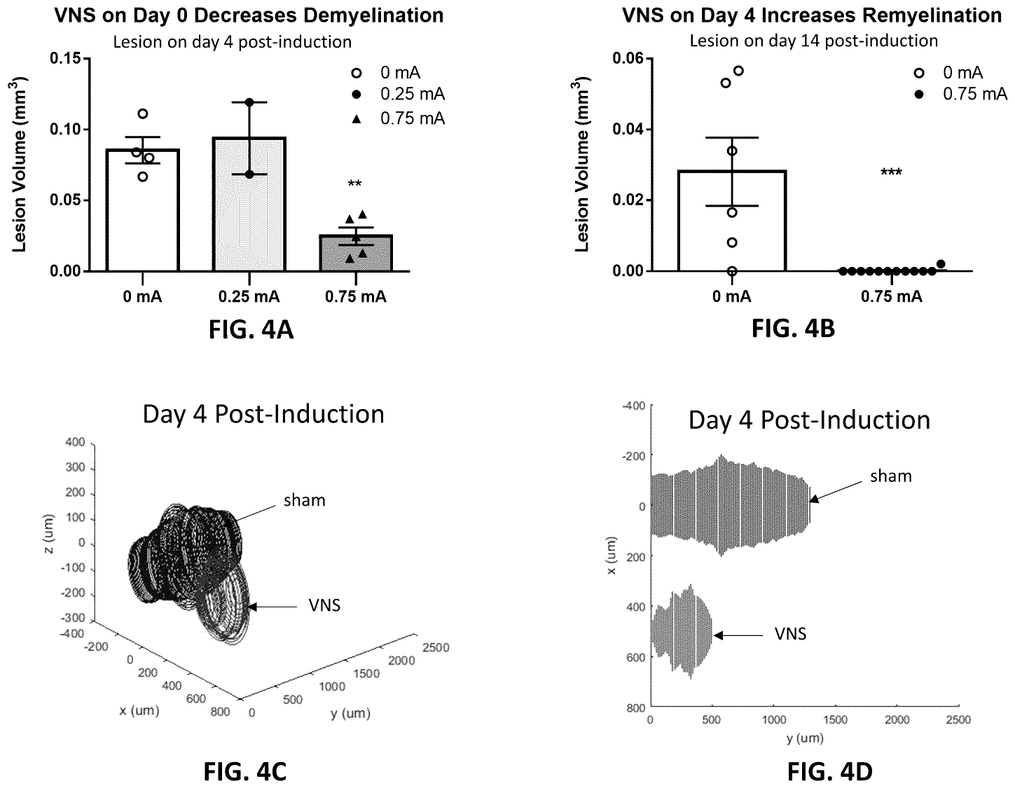

Results: The demyelination protocol illustrated in FIG. 2A showed that VNS inhibited demyelinated lesion progression compared to sham. On day 4 post-induction, the mice were euthanized and the spinal cord around the LPC injection site was sectioned and stained with luxol blue. As shown in FIGS. 4A-4D, the mean lesion volume in the VNS group (0.75 mA) was significantly lower than in the sham group (VNS=0.03 mm3±0.006, n=5, vs. Sham=0.09 mm3±0.009, n=4, p=0.0023). VNS at 0.25 mA resulted in a mean lesion volume similar to sham VNS.

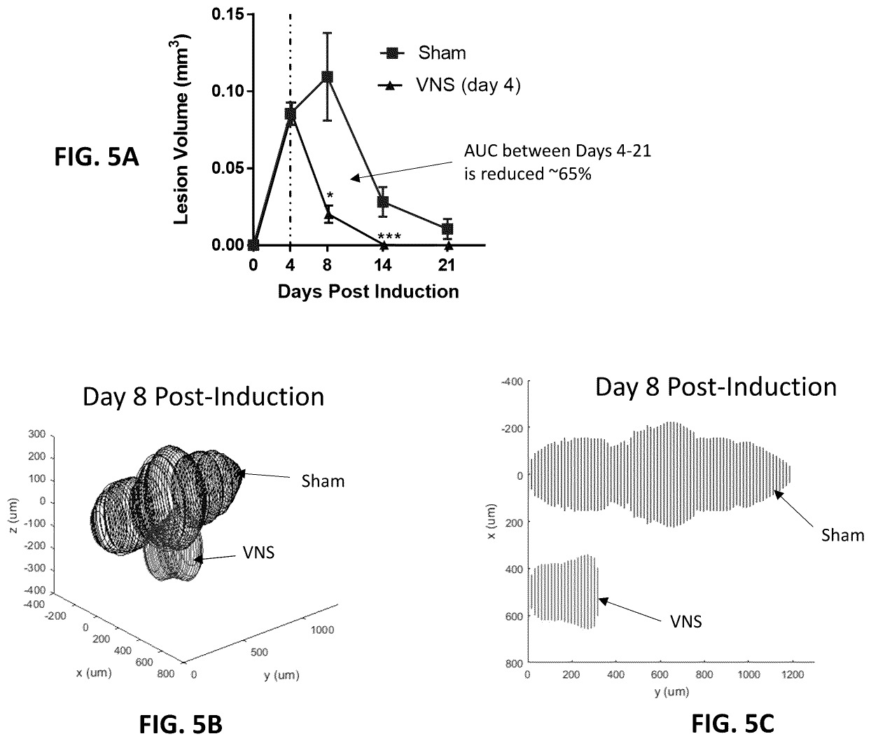

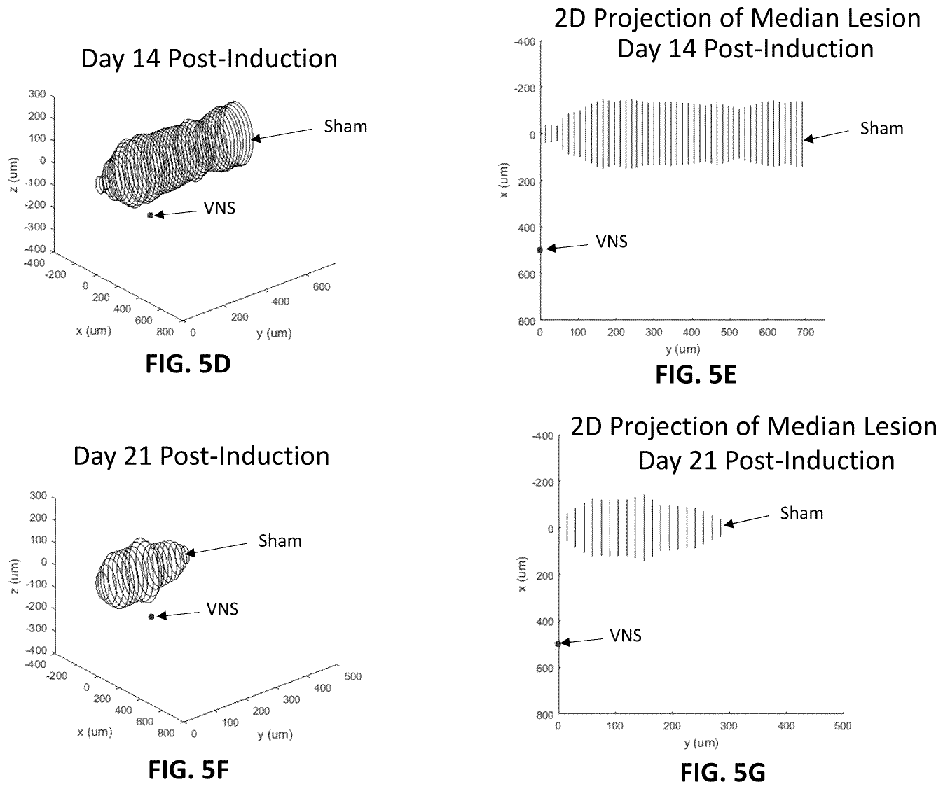

The remyelination protocol illustrated in FIG. 2B showed that remyelination occurred at a significantly accelerated rate in the VNS group. As shown in FIGS. 5A-5G, on day 8 post-induction, mean lesion volume in the VNS group was reduced to 0.02 mm3±0.01, n=4. On day 14 post-induction, mean lesion volume in the VNS group was significantly lower than in the sham group (VNS=0.0002 mm3±0.007, n=12, vs. Sham=0.03 mm3±0.01, n=6, p=0.0007). On day 14, 11 out of 12 VNS animals had no detectable lesion. By Day 21, the mean lesion volume in the sham group was 0.01 mm3±0.006, n=3. FIG. 5A shows that the area under the curve (AUC) between days 4 and 21 is reduced by about 65 percent with vagus nerve stimulation.

Conclusions: VNS reduced demyelination and accelerated remyelination, demonstrating a robust effect after a single dose in this model. Repeated stimulation of the vagus nerve with an implanted nerve stimulator may further reduce the rate of demyelination and/or further accelerate remyelination. This will be tested in an experimental autoimmune encephalomyelitis model to further assess the potential of VNS to treat MS.

### Example 2

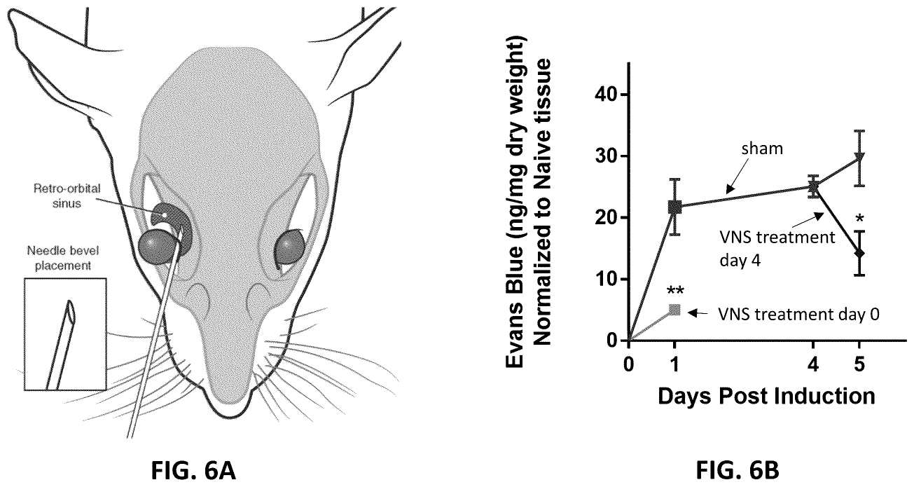

Another study was performed to determine the effect of VNS on vessel leakiness 24 hours post-induction and stimulation. A lesion was induced as described above using LPC injection and VNS was performed immediately following induction. At 24 h, 0.15 mL of 1% Evans blue dye was injected intravenously through retro-orbital injection under anesthesia for 1 hr., as shown in FIG. 6A. One hour later, the animals were euthanized via cervical dislocation. Measurement of extravasation in the spinal cord (SC) was determined by extracting the SC and weighing the SC wet. The SC was then dried for 24 h at 56° C. and weighed dry. The Evans blue dye was extracted with a formamide solvent for 48 h at 56° C. incubation. The supernatant was measured spectroscopically at 620 nm and the quantity of Evans blue dye was determined by interpolation from a reference curve. The quantity of Evans blue dye was normalized to the dry weight of the SC. As shown in FIG. 6B, less Evans blue dye was extracted from the spinal cord from the mice that received VNS, which provides evidence that VNS reduces vessel leakiness 24 hours post-induction and stimulation. In addition, the amount of Evans blue dye extracted from the mice that received VNS was similar to the amount of Evans blue dye extracted from naïve mice (no LPC induced lesion).

Leakiness in the blood brain barrier may allow immune cells and inflammatory cytokines and chemokines to pass through and contribute to continued inflammation in the brain and/or spinal cord. Therefore, VNS may reduce vessel leakiness around the central nervous system (CNS), thereby reducing the recruitment of proinflammatory cells such as lymphocytes (e.g., T-cells) and macrophages to the brain and spinal cord, thereby reducing the inflammation in the CNS and reducing the amount demyelination that results from an inflammatory attack by the immune system.

### Example 3

In general, the apparatuses and methods described for VNS therapy may also be used to prevent or treat increased leakiness of the blood-brain barrier, as illustrate in FIG. 6B.

Methods: 1% LPC was injected into the spinal cord white matter of BALB/c mice. For the first intervention time point, VNS therapy or sham VNS was performed immediately after injection. 24 hours later, mice (VNS, sham VNS, and naïve (no-LPC)) are injected with 1% Evans blue dye which binds to the albumin in blood and is left to circulate for 1 hour. Spinal cords are then harvested, dried for 24 hours in pre-weighed tubes at 60° C. Dried tissues are then incubated in formamide for 48 hours. Supernatant is then extracted from the tubes and read spectroscopically at 620 nm. For the second intervention time point, VNS therapy or sham VNS therapy occurs on day 4 post-LPC induction. On day 5 post-LPC induction, Evans blue extravasation is performed the same way as described for demyelination experiment. Evans blue concentration is compared (ng/mg of tissue) and normalized to naïve animals.

Results: LPC increased blood-spinal cord leakiness. VNS therapy significantly reduced Evans blue extravasation into the spinal cord compared to sham (81% decrease) 24 hours post-LPC induction (FIG. 6B). In addition, VNS therapy on day 4 post-LPC significantly reduced Evans blue extravasation on day 5 compared to sham (52% decrease).

Conclusion: VNS therapy increases the integrity of the blood-spinal cord barrier and subsequently reduces the extravasation of protein/Evans blue and other circulating species, including antibodies, DAMPS/PAMPS, and immunocytes into the central nervous system.

### Example 4

Another experiment was performed to determine whether the effect of VNS on demyelination was α7 nicotinic acetylcholine receptor (nAChR) dependent. Two mice strains were used in the study. One mice strain is the C57 Black subtype 6 (C57BL/6), which is a common wild type strain that expresses α7 receptors and are denoted as α7+/+. The second mice strain is an α7 knockout strain of the C57BL/6 strain, which lacks the α7 receptor and are denoted as α7−/−. Each of the mice strains were given LPC injections in sham (no VNS) and VNS groups. Tissue extraction was performed 4 days post-injection. The procedure was essentially identical to the Balb/c mice demyelination experiments described above in Example 1.

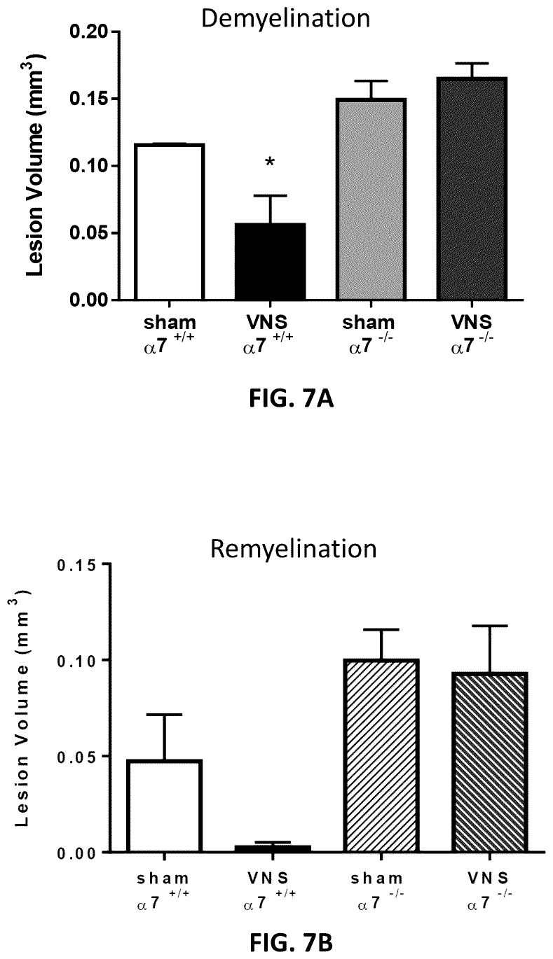

As shown in FIG. 7A, the protective effects of VNS on demyelination is α7 nAChR-dependent. VNS treatment on mice with the α7 nAChR showed a reduced lesion volume when compared with sham, while VNS treatment on mice without the α7 nAChR showed no reduction in lesion volume when compared with sham. Similarly, the remyelination effect of VNS treatment may be α7 nAChR dependent, as shown in FIG. 7B. In this example, the effect of VNS treatment on remyelination in the presence (+/+) and absence (−/−) of the α7 nAChR due to either sham (no VNS treatment) or VNS treatment were examined, showing a substantial decrease in lesion volume, the maker for re-myelination following induction of a demyelination event (e.g., application of LPC.

In FIGS. 7A-7B, 1% LPC was injected into the spinal cord white matter of α7 nAChR knockout mice and C57BL/6 (wildtype) mice. For demyelination experiment, VNS treatment or sham VNS treatment, tissue collection, processing, and analysis are all the same as mentioned above for FIG. 1A. For remyelination, VNS and sham VNS intervention occurs the same as experiment described for FIG. 4B. Spinal cords are harvested only on day 8 post-LPC induction. Processing and analysis performed are the same as described for FIGS. 4A-4B.

Result: VNS therapy decreased demyelination in wildtype C57BL/6 mice. VNS therapy did not decrease demyelination in α7 KO animals (FIG. 7A). VNS therapy increased remyelination in wildtype animals but did not increase remyelination in the knockouts (FIG. 7B). Thus, the effects of VNS on demyelination and remyelination are α7-dependent.

### Example 5

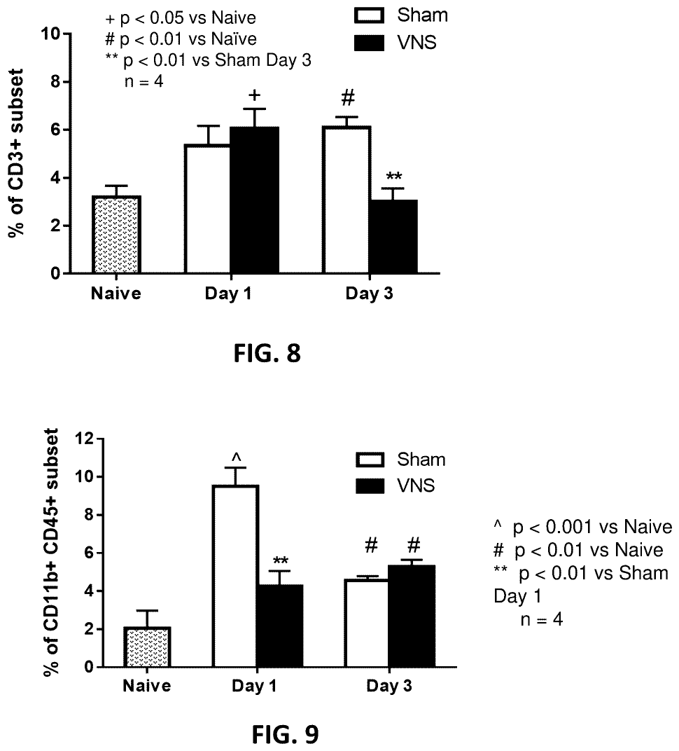

In general, the apparatuses and methods described for VNS therapy may also be used to prevent or treat increased immunocyte homing to the central nervous system, as illustrate in FIGS. 8 and 9.

In FIGS. 8 and 9, CD3+ T cell infiltration through a model for the blood-brain barrier is significantly decreased in the VNS treatment group. As shown in FIG. 8, the CD3+ T cell infiltration through the model of the blood-brain barrier on Day 3 post-LPC induction compared to Sham group is reduced by 50%. In. FIG. 9, the macrophage infiltration is significantly decreased 24 hours post-LPC induction in the VNS treatment group compared to the Sham (no VNS treatment) group by 55%.

Methods: Surgical procedures and VNS/sham VNS treatments remain the same from FIG. 4A. Spinal cords from VNS therapy, sham, and naïve mice are harvested on days 1 or 3 post-LPC induction. Tissue is then digested in enzymatic cocktail for 20 minutes at 37° C. followed by trituration and filtering through a 100 μM mesh screen. Single cell suspension is then put through a density gradient to remove myelin debris from glia cells and immune cells. Once isolated, cells are blocked in FACS buffer and CD32/CD19 for a half hour to prevent unspecific antibody staining. Cells are counted and checked for viability via hemocytometer. Cells are then placed in tubes, stained for either T cells (CD3+) or macrophages (CD11b+, CD45hi) and then analyzed via flow cytometer. Populations of cells are quantified using FlowJo program.

Result: LPC increased CD3+ T cell and macrophage infiltration in the spinal cord compared to naïve tissue (FIGS. 8 and 9). There was a significant reduction in CD3+ T cell infiltration on day 3 post-LPC induction in VNS therapy treated animals compared to sham (50% reduction) (FIG. 9). In addition, VNS therapy resulted in a significant decrease in macrophage infiltration compared to sham 1 day post-LPC induction (55% reduction) (FIG. 9). Thus VNS significantly reduces the infiltration of peripheral immunocytes into the CNS in this lysolecithin-induced MS model.

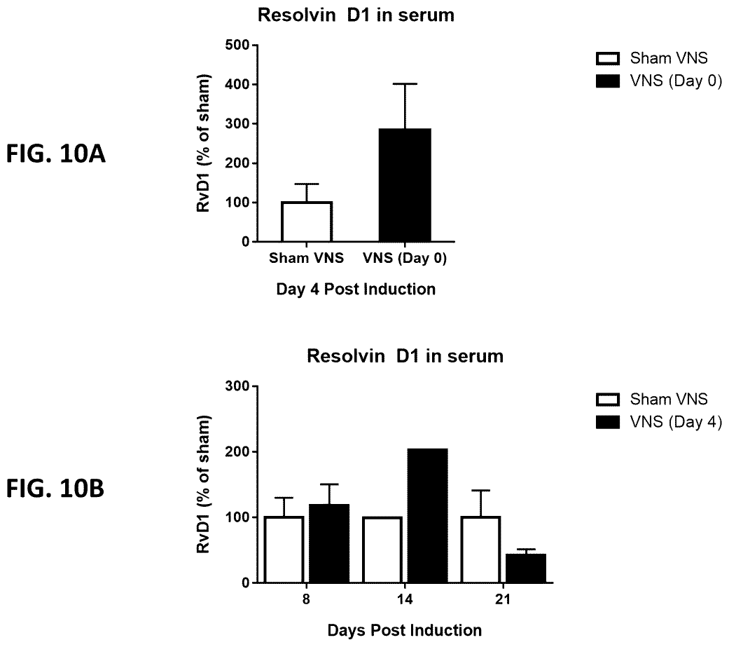

As shown in FIGS. 10A-10B, VNS therapy also increased re-myelination following a decrease in myelination. During spinal cord extractions for all prior experiments performed (see examples 1-4, above), blood was collected via cardiac puncture as well. Blood was centrifuged at 8,000×g for 5 minutes, the serum was collected and stored at −80° C. Using a Resolvin D1 ELISA kit, levels of RvD1 were measured spectroscopically from the serum of VNS and sham VNS mice for the demyelination (D4 harvest) and remyelination (D8, D14, and D21 harvests) experiments. Levels of RvD1 are analyzed (pg/mL) and represented as a percent of sham by day.

Result: as showing FIG. 10A, VNS therapy on day 0 (LPC-induction) increased serum levels of RvD1 on day 4. As shown in FIG. 10B, VNS on day 4 post-LPC induction also increased RvD1 in the serum levels of RvD1 with the highest concentration occurring on day 14 post-LPC induction. RvD1 levels in VNS serum were decreased as compared to sham at 21 days post-LPC induction, likely due to earlier resolution in the VNS group.

Thus, VNS therapy increases the pro-resolving lipid mediator RvD1 in serum which may contribute to the increased speed in resolution time of LPC-induced lesions compared to sham.

### Example 6: System

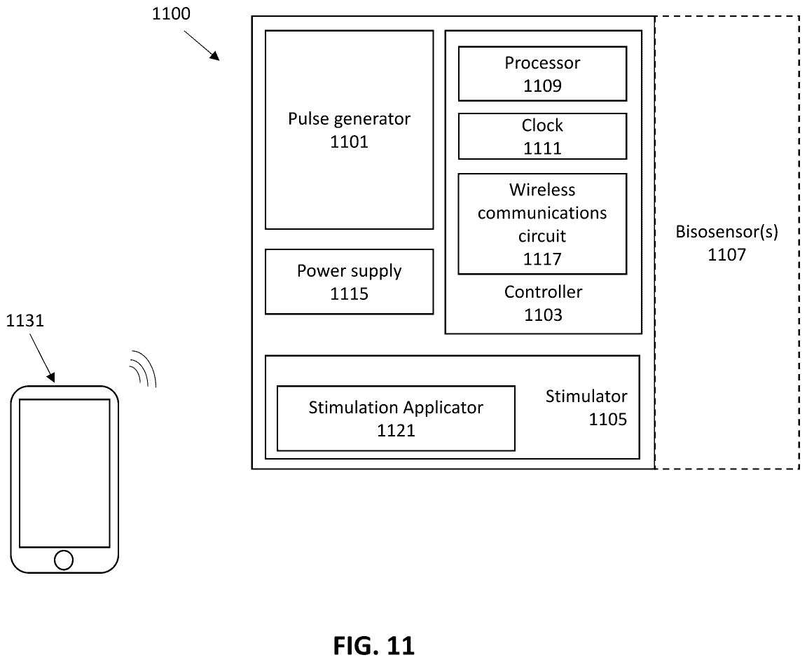

FIG. 11 schematically illustrates one example of a system 1100 for treating demyelination (e.g., for treating MS, or any other demyelinating disorders). In some variations the system for reducing demyelination and/or increase remyelination by stimulation of a vagus nerve includes a controller 1103, a stimulator 1105, and a pulse generator 1101. The pulse generator and stimulator may be connected to and controlled by the controller. In some variations all or some of the system may be implanted into the patient's body. All or some of the components of the apparatus may be enclosed by a housing (e.g., an implant housing). In general, the systems may also include one or more biosensor 1107 configured to detect one or more biomarkers. The biosensor may be coupled with the rest of the system (e.g., implant) or it may be separate and may communicate via a wired or wireless connection. For example the biosensor may be implanted into the body so as to sample blood, spinal fluid, or the like; in some variations the biosensor is external to the body and may be single use or configured for limited-reuse. In some variations the biosensor may include a sensor for determining a patient's physical condition (e.g., temperature, nerve conduction, etc.). In some variations the biosensor may be an immunochemical sensor configured to detect binding of one or more analytes and/or to provide a concentration.

The stimulator may be configured to apply stimulation to the vagus nerve. A stimulator may be configured for electrical stimulation, mechanical stimulation, or both. For example, the stimulator may include or be coupled with the pulse generator 1101 (e.g., waveform and/or pulse generator, oscillator, etc.). The stimulator may include one or more stimulation applicators 1121 (e.g., one or more electrodes, mechanical transducers, etc.) for contact with the tissue, including the vagus nerve.

Any of the apparatuses may also include one or more power supplies 1115, and/or power regulation circuit, etc.

The controller is typically functionally coupled to the one or more biosensor (e.g., receiving data from the biosensor(s)) and controls the stimulator and may be configured to apply stimulation to the vagus nerve from the stimulator sufficient to reduce demyelination and/or increase remyelination of nerves within the patient when the biosensor detects a biomarker indicative of demyelination (including detecting active demyelination or a marker that is indicative of imminent active demyelination).

For example, a system may include an implant comprising a stimulator (e.g., a waveform and/or pulse generator, an oscillator, a power supply and/or power regulation circuit, etc.), a stimulation applicator (e.g., one or more electrodes, mechanical transducers, etc.), and a controller. The controller may be configured as a microcontroller and may be in electrical communication with the stimulator so as to control operation of the stimulator. The controller may include one or more processors, a memory and/or a timer. The stimulator and/or controller may be in electrical communication, one or more stimulation applicators. In some variations the controller may include or be in communication with wireless communications circuitry 1117 for wirelessly communicating with one or more remote processors 1131. The remote processor may be a hand-held device (e.g., smartphone, wearable electronics, etc.). The controller may optionally be in communication with one or more biosensors that may be included with the implant or may be remote from the implant (e.g., may be wearable, single-use, etc.). In some variations the biosensors are wirelessly connected to the apparatus.

In any of the apparatuses and methods described herein, the apparatus may be configured to deliver a limited range of charge per day to improve MS, as descried herein. For example, these methods and apparatuses for treating MS (or otherwise reducing or preventing demyelination and/or for increasing remyelination by stimulation of a vagus nerve) may be configured to be implanted over or adjacent to a vagus nerve to apply electrical stimulation to the vagus nerve in which a controller coupled to the vagus nerve stimulator is configured to apply electrical stimulation to the vagus nerve from the one or more electrodes, wherein the controller is constrained to apply a charge per day of between about 2.5 nC and 7.5 mC to reduce demyelination and/or increase remyelination within the patient, and thereby treat MS. This apparatus may be system.

For example, the controller may be configured to deliver the electrical stimulation during one or more dose sessions of about 5 minutes or less (e.g., 4 min or less, 3 min or less, 2 min or less, 1 min or less, etc.). The controller may be configured to apply the charge per day at a frequency of between 1 and 20 Hz. In some variations the controller is configured to apply the charge per day at a frequency of between 1 and 12 Hz. Thus, these methods and apparatuses may include the application of vagus nerve stimulation within a range of charge (e.g., in nanocolumb to microcolumb range) of between about 2.5 nC/day (e.g., about 0.1 mA and 0.1 msec pulse-with VNS) to about 7.5 mC/day. Pulses may be applied between 0.1 and 50 Hz (e.g., between 1 and 20 Hz, etc.). The charge may be delivered either directly, e.g., by an implantable device, or indirectly, as by a transcutaneous delivery device. Outside of these ranges (e.g., the application of less than 2.5 nC/day) typically has little or no effect on patients. Similarly, the application of greater than about 7.5 mC/day may have no additional effect and in some cases may result in an inhibition of the effect. Thus it may be beneficial to limit the daily application of charge to be between about 2.5 nC and 7.5 mC per day (e.g., between about 5 nC and 7 mC, between about 10 nC and about 6.5 mC, between about 50 nC and about 6 mC, between about 100 nC and about 6 mC, etc.).

### Example 7: Confirmation in an EAE Model

As discussed above, multiple sclerosis (MS) is an inflammatory, demyelinating disorder of the central nervous system (CNS) that afflicts approximately 900,000 adults in the United States alone and is associated with axonal damage, impairment in neurologic function, and progressive disability. The etiology of MS is incompletely defined, although an interplay of genetic and environmental factors is thought to underlie the disorder. From a pathophysiological perspective, astrocyte activation and CNS influx of autoreactive lymphocytes (eg, CD4+ Th1 and Th17 cells that secrete IFN-γ, IL-12, and IL-17) and other inflammatory mediators (eg, the plasma protein fibrinogen) across a disrupted blood brain barrier (BBB) are associated with initiation and perpetuation of demyelination. Drug therapies approved for the treatment of MS have been shown to inhibit CNS inflammation and slow progression of disability, however, these agents neither work well for, nor are they tolerated by, every MS patient; serious adverse events can preclude or limit their use. There is a significant need for new treatments that are efficacious and well tolerated. Recent advances in bioelectronic medicine indicate that non-pharmacologic approaches may be less immunosuppressive and have fewer off-target effects.

One such approach described herein is the electrical activation of the vagus nerve (VN). The VN is the longest cranial nerve, extending bidirectionally between the brainstem and the viscera. Due to its accessibility in the cervical region and connections with brain centers of clinical interest, the VN has long been a target for neuromodulation in CNS indications. VNS using specific stimulation parameters has been demonstrated to reduce inflammation, symptoms of disease, and tissue damage in multiple animal models and clinical trials of peripheral and central inflammation-driven disorders (neuro-immunomodulation). Termed the “Inflammatory Reflex”, the VN provides a neural mechanism to sense peripheral inflammation and reflexively regulate innate immunity by reducing proinflammatory cytokines. Information regarding presence of peripheral cytokines is sent via afferent vagus axons to the brain, which responsively sends signals through efferent VN fibers. Neuronally-derived norepinephrine (NE), which binds to β2 adrenergic receptors on tissue-resident lymphocytes (eg, in the spleen), stimulates ChAT+ lymphocytes to release ACh. Neuron- and lymphocyte-derived ACh binding specifically to α7 nicotinic acetylcholine receptors (α7nAChR) on tissue-resident and circulating immunocytes leads to altered immunocytic phenotype, typified by a decrease in proinflammatory cytokine release, downregulation of inflammatory cell trafficking and extravasation into tissues, and protection against inflammation-mediated damage. Intracellular signaling cascades downstream of α7nAChR activation include Nf-κB, inflammasome, and the JAK/STAT pathways and lead to downregulation of inflammation.

Surprisingly, as described herein, VNS may be an effective treatment for MS. Specifically, subacute VNS may be used. This was tested in a standard rodent model, experimental autoimmune encephalomyelitis (EAE). We observed that VNS decreased the severity and duration of EAE, reduced inflammatory lesion formation and demyelination, decreased BBB disruption and CNS entry of neutrophils and pathogenic lymphocytes (Th1/Th17), reduced fibrinogen deposition, shifted microglia/macrophage phenotype toward repair, and modulated gene expression of IL-12, IFN-γ, IL-17, inflammatory reflex components, as well as myelin synthesis.

### Results

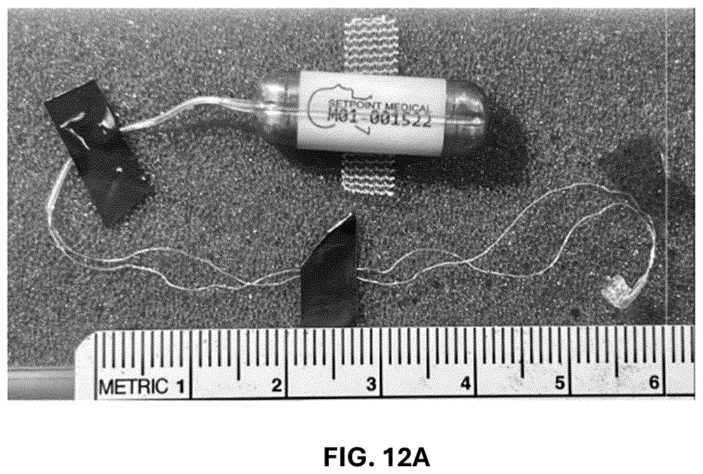

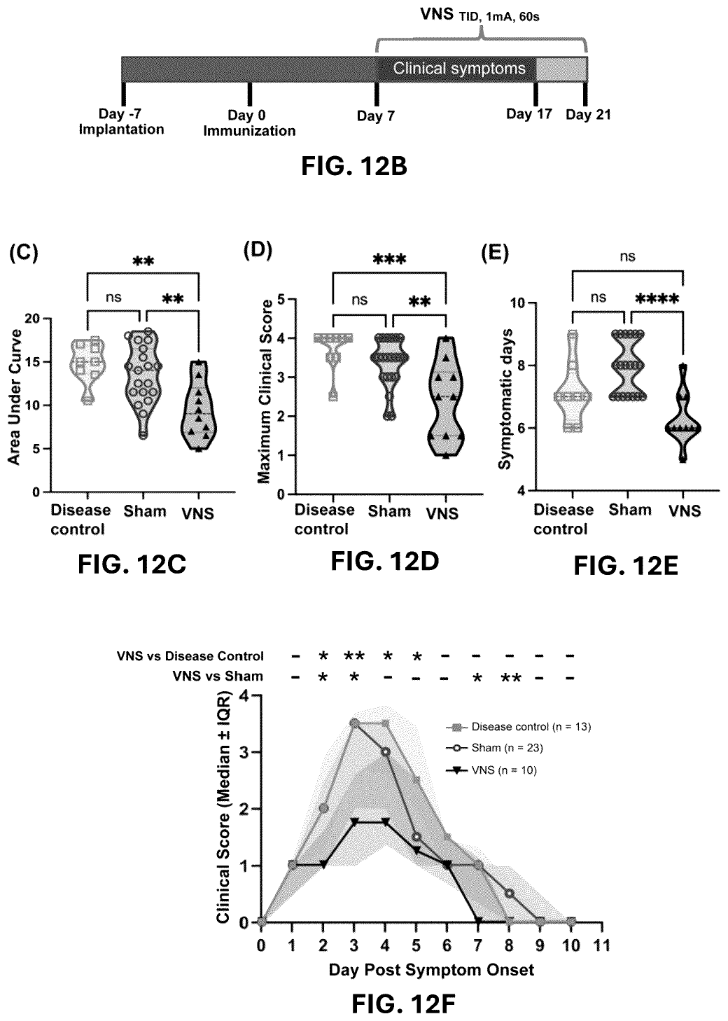

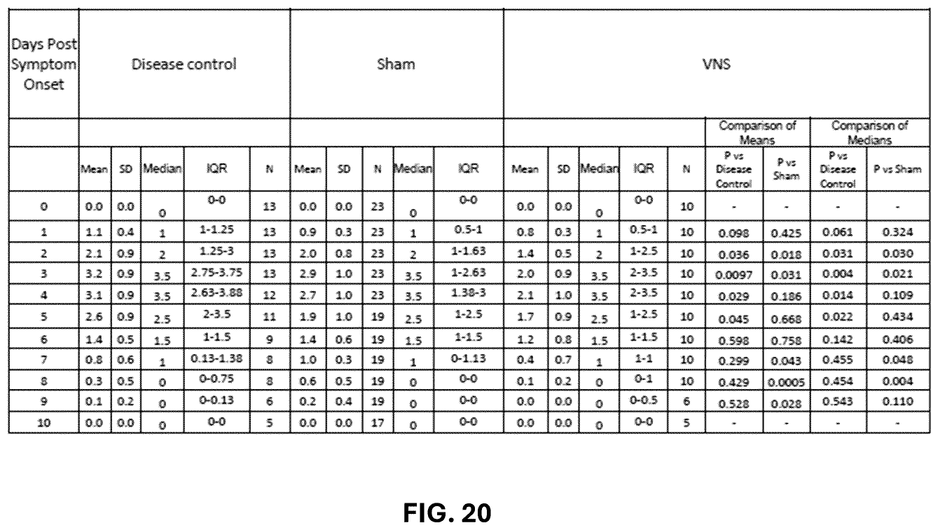

VNS reduces disease severity and duration in EAE: To investigate whether VNS is effective in reducing EAE severity and duration, device (table 2, shown in FIG. 20), showing implanted rats were stimulated daily (1 mA, 60 s, TID), beginning 7 days post-EAE induction (DPI), the approximate day of EAE symptom onset, through day 21 DPI (FIG. 12B A). For analysis of grouped data, individual animals were re-baselined to the day post symptom onset (DPSO). VNS significantly reduced EAE disease severity and duration compared with Sham (device implanted, no stimulation) and unimplanted disease control rats (FIG. 12B B, and table 2); by 2-way mixed model ANOVA: treatment p=0.005, DPSO p<0.0001).

VNS significantly attenuated EAE disease severity and duration, as demonstrated by data representing area under the curve (AUC=clinical score X number of symptomatic days; FIG. 12C-12F; mean±SEM; disease control=14.88±0.80, Sham=13.50±0.76, and VNS=9.45±1.01, p=0.0013 vs Sham), maximum clinical score (FIG. 12D; mean±SEM: disease control=3.72±0.17, Sham=3.36±0.13, and VNS=2.4±0.31, p=0.0005 vs. Sham) and number of symptomatic days (FIG. 12E; mean±SEM: disease control=7.13±0.35, Sham=7.95±0.19, and VNS=6.30±0.26, p=0.0001 vs. Sham). Consistent with reduction in disease severity and duration, VNS ameliorated the weight loss that was observed in Sham and disease control rats (Table 3; by 2-way ANOVA: treatment p=0.003, DPSO p<0.0001).

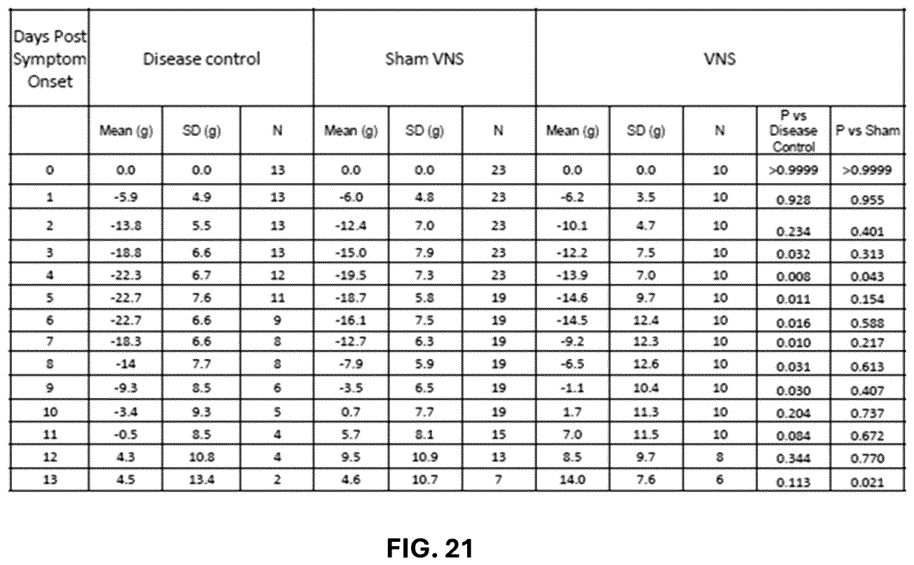

Moreover, the magnitude of VNS suppression of EAE disease severity and duration was not significantly different from the effect achieved with 3 mg/kg/day of the FDA-approved oral MS drug, teriflunomide (Table 3, FIG. 21; AUC mean±SEM: Vehicle=14.38±1.43, 1 mg/kg/day teriflunomide=12.75±1.64, 3 mg/kg/day teriflunomide=7.00±2.10, VNS=9.45±1.01, p=0.594 vs. 3 mg/kg/day teriflunomide).

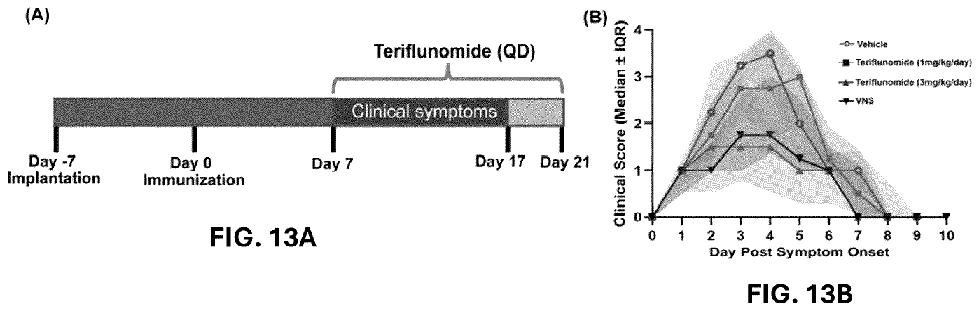

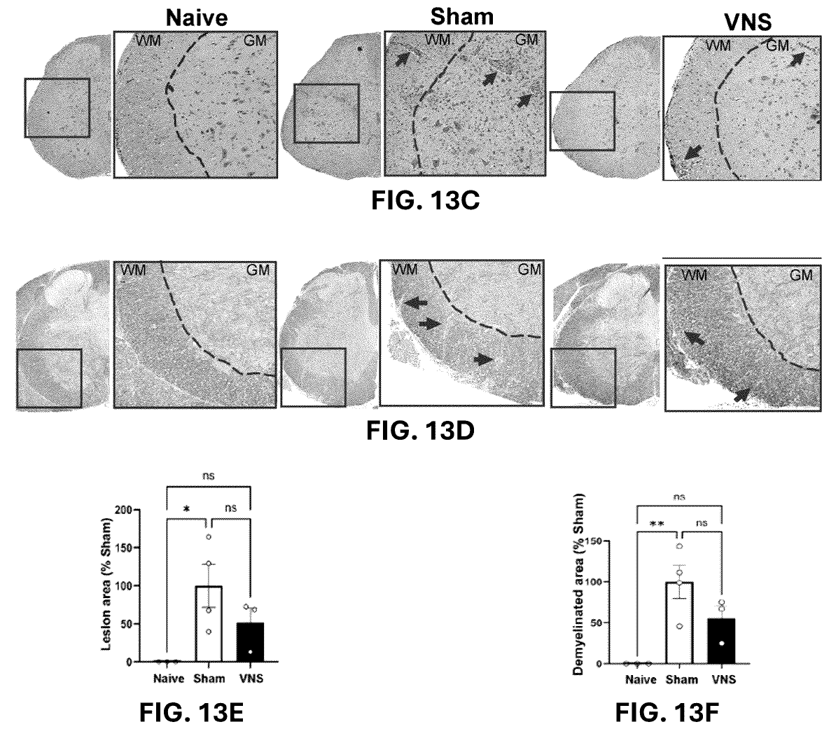

VNS reduces inflammatory lesion formation and demyelination in EAE spinal cord (SC) at peak disease: To explore mechanisms underlying VNS-mediated reduction in disease severity and duration, histochemistry analyses were utilized to assess inflammatory lesions and demyelination of VNS, Sham, and naïve rat lumbar SC at 3-4 days DPSO (Peak EAE) (FIG. 13A). As expected, there were no abnormal findings in naïve rat SC by hematoxylin and eosin (H&E) or Luxol fast blue (LFB) with nuclear fast red staining (FIGS. 13C-13D). H&E staining at 3-4 DPSO revealed that VNS reduced total inflammatory lesion area by 49% compared with elevated levels observed in both white matter (WM) and grey matter (GM) SC regions (arrows) of Sham rats (FIGS. 13C and 13E; mean % Sham±SEM: Sham=100.00±28.36, VNS=51.33±19.20, Naïve=0.00±0.00; n=3-4 per group; Sham vs. VNS: p=0.33, Sham vs. Naive: p=0.04, VNS vs. Naive: p=0.34). These numerous WM and GM lesion areas colocalized with cellular infiltrates, providing evidence that VNS prevented infiltration of lesion-associating immunocytes (FIG. 13C). LFB staining at symptom peak revealed that VNS reduced the extent of demyelination in WM by 44% compared with Sham, with several observed VNS SC segments completely free of perceptible demyelination (FIGS. 13D and 13F; mean % Sham±SEM: Sham=100.00±20.35, VNS=55.72±15.55, Naïve=0.00±0.00; n=3-4 per group; Sham vs. VNS: p=0.20, Sham vs. Naive: p<0.001, VNS vs. Naive: p=0.13). Higher levels of both cellular infiltration-associated lesions and demyelination were also observed in Sham vs. VNS and naïve rats at 0-2 DPSO (Worsening EAE), and 5-7 DPSO (Remitting EAE) timepoints (FIG. 13C-13F).

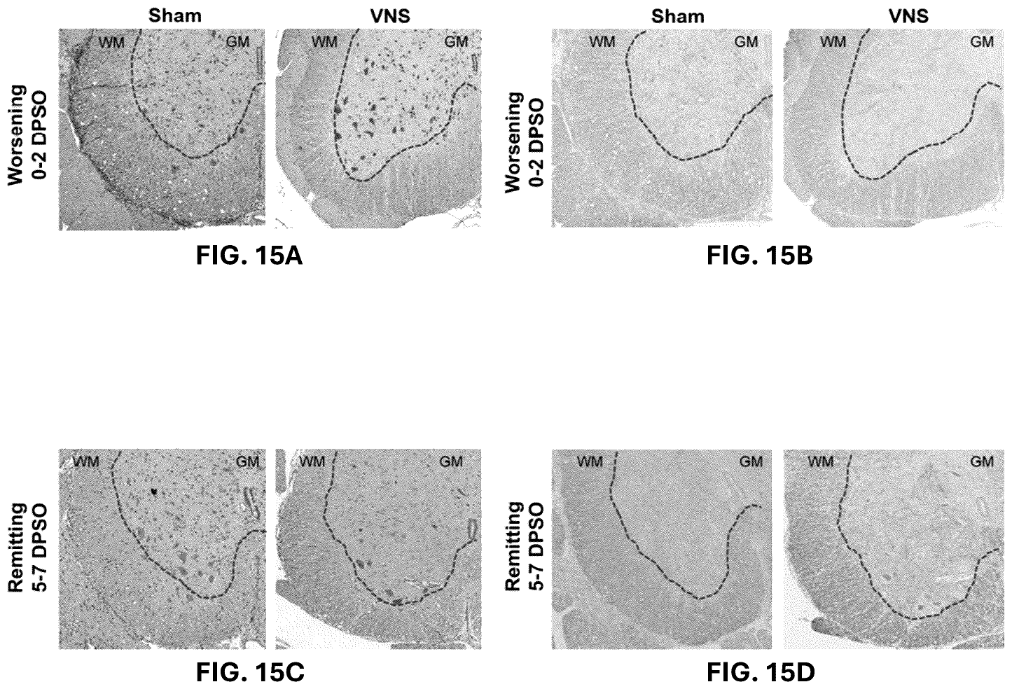

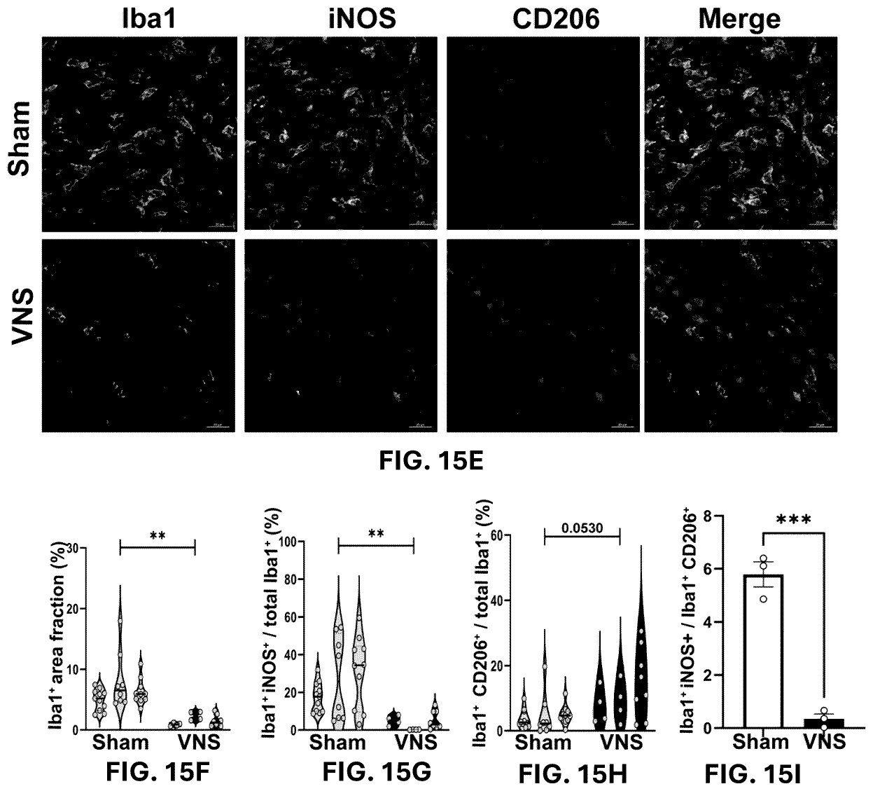

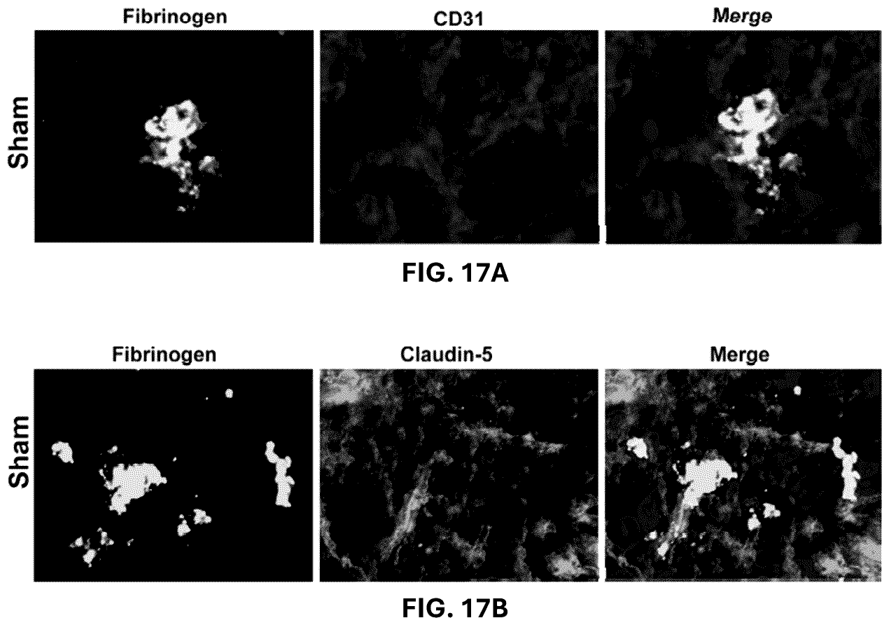

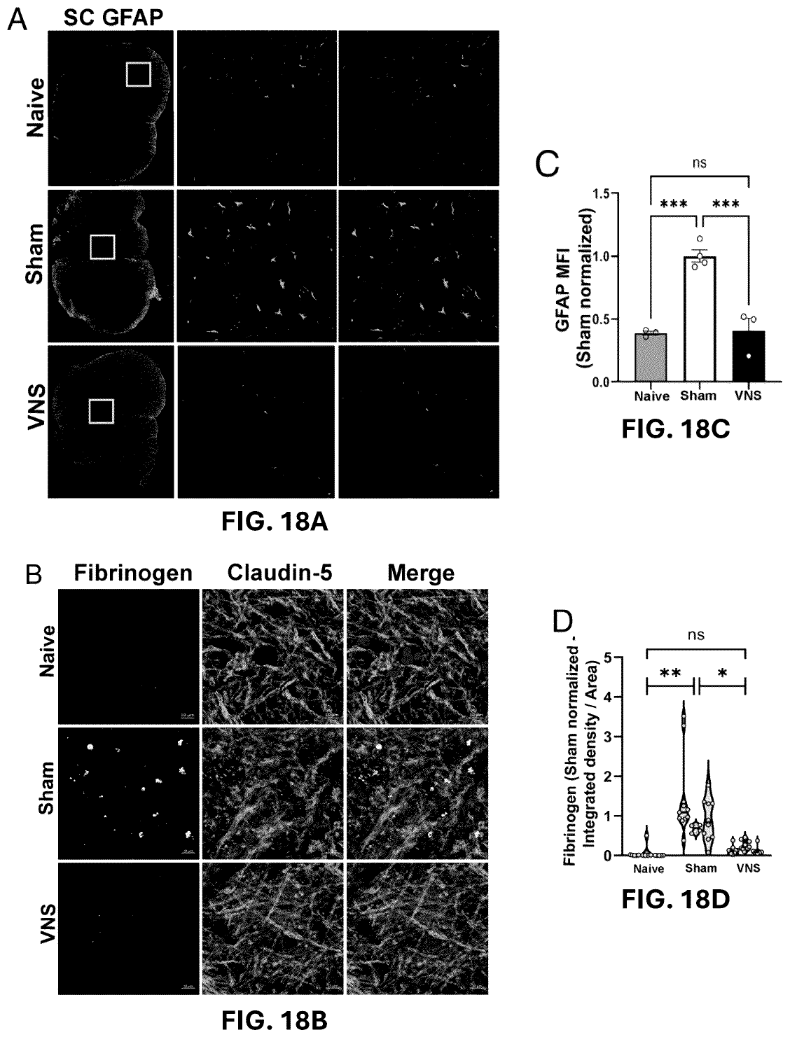

VNS suppresses activation of astrocytes, preserves BBB integrity, and restricts fibrinogen deposition at peak EAE: Immunofluorescence studies were conducted to investigate the presence of astrocyte activation, BBB disruption, and parenchymal deposition of fibrinogen. The extent of BBB-destabilizing astrocyte activation during EAE was interrogated with glial fibrillary acidic protein (GFAP) staining. The Sham group displayed significantly higher GFAP expression relative to VNS throughout the GM and WM regions (FIGS. 18A and 18C). The reduction in GFAP staining indicated that VNS reduced astrocyte activation (GFAP mean fluorescence intensity (MFI) normalized to Sham: Sham=1.00±0.05, VNS=0.407±0.1, Naïve=0.388±0.15; Sham vs. VNS: p=0.0006, Sham vs. Naïve: p=0.0005, VNS vs. Naïve: p=0.98). VNS maintained distribution of CNS endothelial tight junction protein claudin-5 in a defined mesh-like appearance, in contrast to the dysregulated expression observed in Sham (FIG. 18B). CNS parenchymal deposition of the plasma protein fibrinogen was highly evident in Sham SC segments (FIGS. 15B and 15D), which was elevated in both WM and GM and occasionally colocalized with areas of endothelial cell markers claudin-5 and CD31 disruption (FIGS. 18B and 18D, and FIGS. 17A-17B). In contrast, evident deposition of fibrinogen was scarce in VNS SC (FIGS. 18B, 18D), with the density of fibrinogen staining significantly reduced in VNS versus Sham SC (mean normalized to Sham±SEM: Sham=1.00±0.22, VNS=0.17±0.04, Naïve=0.04±0.03; n=3; Sham vs. VNS: p=0.010, Sham vs. Naive: p=0.005, VNS vs. Naive: p=0.79). Together, these data suggest that VNS treatment suppresses astrocyte activation, preserves the integrity of the BBB, and limits deposition of fibrinogen into the CNS parenchyma.

VNS suppresses activation of macrophage/microglia and shifts their phenotype toward resolution at peak EAE: Immunofluorescence studies were conducted to investigate the activation state of both lesion-adjacent and lesion-distal microglia/macrophage by co-labeling microglia/macrophage marker Iba1 with the proinflammatory “M1” phenotype marker iNOS and the anti-inflammatory/pro-resolving “M2” phenotype marker CD206. Iba1+ area was significantly reduced at the peak of EAE disease in VNS compared with Sham rats (mean Iba1+ area fraction (%): Sham=6.462±0.898, VNS=1.562±0.411; n=3, p=0.008), suggesting a suppression of microglial activation and proliferation (FIGS. 15E-15I). Co-staining of Iba1 with the “M1” and “M2” markers indicates that VNS significantly skews microglia from predominantly iNOS-expressing toward CD206-expressing phenotype (mean % iNOS-expressing microglia: Sham=25.45±4.14, VNS=3.53±1.66; n=3, p=0.008, mean % CD206-expressing microglia: Sham=4.33±0.39, VNS=10.47±2.22; n=3, p=0.053, mean ratio between Iba1+iNOS+ and Iba1+CD206+: Sham=5.79±0.47, VNS=0.35±0.18; n=3, p=0.0004).

VNS Reduced Entry of Pathogenic Immunocytes into the CNS:

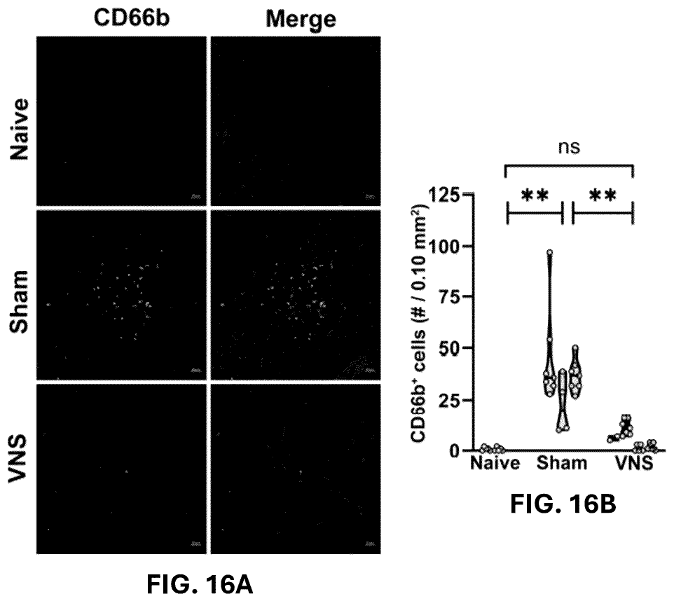

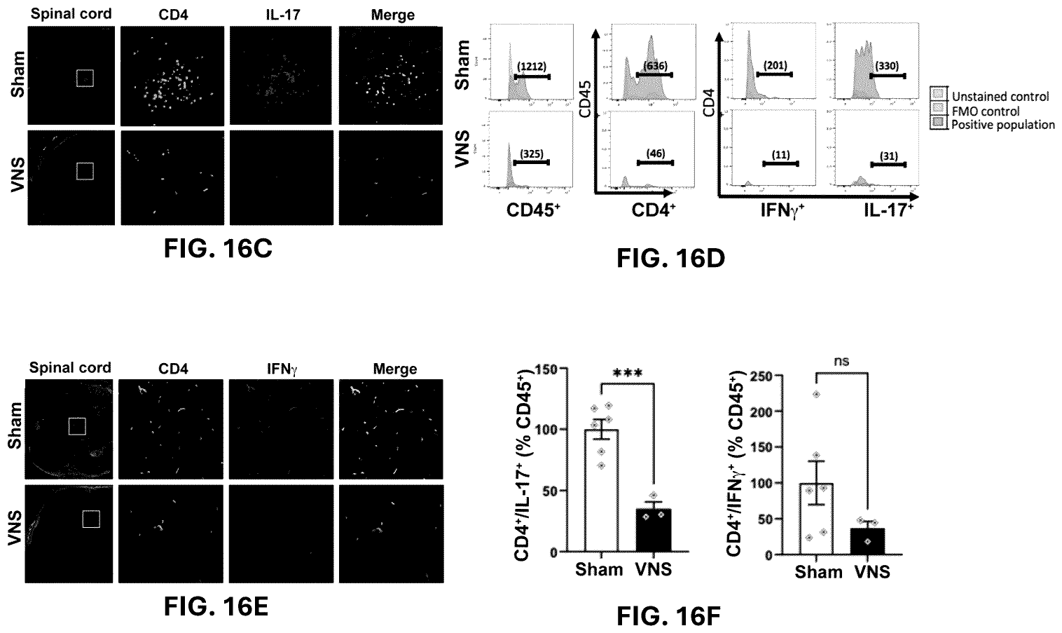

Neutrophil infiltration into the CNS during the initial days of symptomatic disease was determined by immunofluorescent staining of CD66b positive cells on 0-2 DPSO. The number of CD66b+ neutrophils was significantly reduced in the VNS group as compared to the Sham group (#CD66b+ cells/focal area±SEM: Sham=34.94±6.81, VNS=5.16±2.34, Naïve=0.70±0.10; n=2-4; Sham vs. VNS: p=0.005, Sham vs. Naive: p=0.006, VNS vs. Naive: p=0.78) (FIGS. 16C-16D). Immunofluorescence and flow cytometry analyses were employed to determine whether the cellular infiltrates observed in SC at peak EAE (3-4 DPSO) were comprised of lymphocytes known to be pathogenic in MS and EAE. Immunofluorescent staining of SC sections with antibodies recognizing T lymphocyte marker CD4 and cytokines IL-17 and IFN-γ showed greater numbers of IL-17+ and IFN-γ+ CD4+ T-cells in Sham than in VNS (FIG. 16A-16B). Flow cytometry analysis of isolated SC cells stained for leukocyte marker CD45, as well as CD4, IL-17 and IFN-γ, also showed a 64.9±5.6% reduction in numbers of CD45+/CD4+/IL-17+ (% CD45+ population and normalized to Sham; mean±SEM, p=0.0003) and 63.0±9.0% reduction in numbers of CD45+/CD4+/IFN-γ+ (% CD45+ population and normalized to Sham, mean±SEM, p=0.095) lymphocytes in VNS rats compared to Sham rats (FIGS. 16A-16F). Together, these findings suggest that VNS inhibits lesion formation and demyelination by reducing pathogenic cellular infiltration into the CNS.

### VNS Modulates Expression of Genes Involved in Th1 and Th17 Inflammatory Pathways, Key Inflammatory Reflex Components, and Myelin Synthesis:

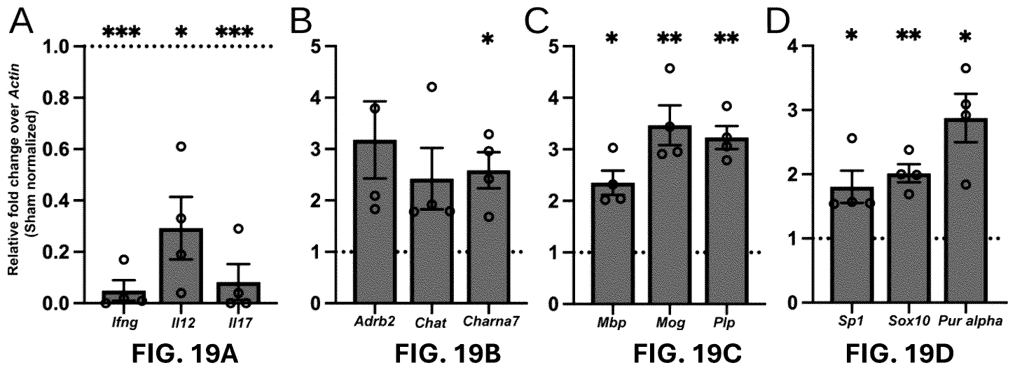

The EAE disease course of the VNS group diverged from that of disease control and Sham within 24-72 hours of VNS initiation (0-2 DPSO; FIG. 12F). To explore how VNS impacts key pathways underlying EAE pathology, quantitative polymerase chain reaction (qPCR) was performed on signature genes of the inflammatory reflex, genes involved in Th1 and Th17 inflammatory pathways, and genes involved in myelin synthesis and oligodendrocyte differentiation in SC at 0-2 DPSO. Gene expression levels of Ifng, Il12, and Il17, inducers of pathogenic Th1 and Th17 activity, were all significantly downregulated in VNS rats (mean fold change from Sham±SEM: Ifng: 0.05±0.04, n=4, p=0.0002; 1112:0.29±0.12, n=4, p=0.01; Il17: 0.08±0.06, n=4, p=0.001) (FIG. 17A). Cytokine gene expression levels of Ifng, Il12 and Il17 were consistent with a protective effect of VNS in EAE. Expression of α7 nicotinic acetylcholine receptors (Charna7) gene was significantly increased (mean fold change from Sham±SEM: 2.59±0.35, n=4, p=0.02) while gene expression levels of β2 adrenergic receptors (Adrb2) and choline acetyltransferase (Chat) trended upward (mean fold change from Sham±SEM: Adrb2: 3.180±0.75, n=4, p=0.062, Chat: 2.425±0.59, n=4, p=0.0966) (FIG. 17 B). Gene expression levels of myelin basic protein (Mbp), myelin oligodendrocyte glycoprotein (Mog), and proteolipid protein (Plp), which encode several major protein components of myelin and are associated with oligodendrocyte differentiation and new myelin synthesis, were all significantly upregulated in VNS rats (mean fold change from Sham±SEM: Mbp: 2.35±0.24, n=4, p=0.0105; Mog: 3.47±0.39, n=4, p=0.0078, Plp: 3.23±0.22, n=4, p=0.0021) (FIGS. 19A-19D). The transcription factors specificity protein 1 (Sp1), SRY-box transcription factor 10 (Sox 10) and Pur-α (Pur alpha), which bind at the Mbp promoter region were all significantly upregulated in VNS rats (mean fold change from Sham±SEM: Sp1: 1.81±0.25, n=4, p=0.049; Sox 10:2.02±0.14, n=4, p=0.006, Pur alpha: 2.88±0.38, n=4, p=0.0158) (FIG. 19D). Gene expression data suggest that VNS may enhance oligodendrocyte differentiation, myelin production, and inflammatory reflex signal transduction.

These data collectively indicate that VNS treatment decreased rat EAE disease severity and duration by preventing pathological neutrophil and lymphocyte infiltration, myelin damage, blood-brain barrier disruption, and fibrinogen deposition, skewed microglia/macrophage toward repair phenotype, and beneficially modified expression of genes encoding mediators of inflammation, the inflammatory reflex, and myelin synthesis.

Treatment with VNS decreased the severity and duration of disease in a standard rat EAE model of MS. VNS reduced astroglial activation, skewed microglia/macrophage toward resolution phenotype, maintained BBB integrity, decreased parenchymal fibrinogen deposition, and modulated the expression of genes encoding components of secreted inflammatory mediators, inflammatory reflex signaling, and myelin synthesis. VNS also decreased CNS infiltration of neutrophils and pathological T lymphocytes and reduced the extent of inflammatory lesions and demyelination. Furthermore, in a direct comparison with the FDA-approved oral disease-modifying therapy (DMT) teriflunomide, the magnitude of reduction in EAE disease severity and duration achieved by VNS mirrored that attained by teriflunomide, consistent with a previous publication that studied teriflunomide in the same EAE Lewis rat model. The EAE model has also provided predictive validity in the development of many drugs approved for the relapsing remitting form of MS, which similarly have demonstrated efficacy in EAE.

VNS has been established as a safe treatment for drug-resistant epilepsy, depression, and ischemic stroke-associated motor deficits, with VNS devices implanted in >125,000 patients. We and others have demonstrated the potential for VNS to access the inflammatory reflex to non-pharmacologically reduce symptoms and disease pathology, and even in some cases recover function, in a wide variety of animal models and clinical trials of inflammation-driven diseases. Several small prospective clinical studies of VNS as a treatment for the autoimmune disorders rheumatoid arthritis and Crohn's disease have reported positive results, and a large double-blind pivotal study of VNS in subjects with drug-resistant rheumatoid arthritis is currently underway (RESET-RA; NCT04539964).

While no animal model perfectly replicates human disease, EAE recapitulates many of the pathophysiologic and clinical properties of MS. In this study, we observed therapeutic benefit of VNS on several key features of the EAE model. In both MS and EAE, loss of myelin is associated with formation of WM and GM lesions, impairment in neurologic and cognitive function, and mood disturbances such as depression. In our study, VNS reduced focal areas of WM and GM lesions and areas of WM demyelination by 49% and 44%, respectively. While these area reductions assessed at a single timepoint (3-4 DPSO) and with relatively small sample size did not reach statistical significance, evaluation of clinical symptoms across the entire observation period revealed that VNS treatment significantly attenuated both severity and duration of EAE, which is also reflected by a corresponding significant attenuated loss of body weight.

In MS and EAE, autoreactive lymphocytes and secreted mediators damage components of the myelin sheath (e.g., MBP, PLP, and MOG) and the oligodendroglia that generate CNS myelin. Astrocytes in active lesions become hypertrophic and express high levels of activation marker GFAP as well as proinflammatory cytokines and chemokines that lead to and exacerbate BBB disruption. BBB integrity also depends on support from pericytes, endothelial cells and the end-foot processes of astrocytes; loss of astrocytic end-foot contact with pericytes and endothelial cells and disruption of glial-blood vessel interactions via multimeric gap and tight junction proteins (e.g., claudin-5) lead to enhanced vascular permeability. Enhanced BBB permeability facilitates CNS infiltration of normally excluded myelin-reactive immunocytes (in particular, autoreactive CD4+ Th1 and Th17 cells that produce IFN-γ, IL-12, and IL-17), neutrophils, activated macrophages, dendritic cells, and plasma proteins such as fibrinogen. Extravascular deposition of fibrinogen triggers chemokine release and initiates inflammatory demyelination in the CNS, and fibrinogen has also been identified in CNS lesions as both biomarker and driver of disease in MS and EAE.

Data from the current study indicate that VNS decreased BBB disruption and myelin damage when assessed at 3-4 DPSO (Peak EAE). VNS suppressed activation of astrocytes and showed patterns of claudin-5 immunostaining suggestive of maintenance of normal BBB integrity. This was substantiated by a significant reduction in parenchymal fibrinogen deposition in VNS-treated rat SCs. Both qualitative (immunofluorescence staining) and quantitative (flow cytometry) analyses showed reductions in numbers of pathogenic T-lymphocytes that colocalized with GM and WM lesions (statistically significant for CD45+/CD4+/IL-17+, numerically for CD45+/CD4+/IFN-γ+). It is well established that pathogenesis of both MS and EAE is associated with both Th-1 and Th-17 subsets of T cells, through their secretion of cytokines such as IFN-γ, IL-12, and IL-17. IL-12 is also implicated in the propagation of additional IFN-γ producing Th-1 cells. Expression of the proinflammatory cytokine genes Ifng, Il12, and Il17 are respectively linked with these Th-1 (IL-12, IFN-γ) and Th-17 (IL-17, IL-23) cytokine pathways. The observed downregulated expression of Ifng, Il112 and Il17 in EAE therefore supports the therapeutic potential of VNS in MS.

Inflammatory activation of innate immune cells in the CNS is implicated in MS pathogenesis. Release of proinflammatory cytokines and chemokines from Iba1+/INOS+ resident microglia and infiltrated macrophage lineage cells recruit pathogenic lymphocytes to the CNS, while production of high levels of reactive oxygen species (ROS) damage local tissues, including the myelin sheaths that surround and protect neuronal axons. Yet, just as these cells mediate CNS damage, microglia/macrophages are also required for normal resolution of the damage caused by inflammation, including remyelination. Following a course of damaging inflammation, healthy resolution is mediated by a phenotypic shift in these cells from M1 Iba1+/iNOS+ proinflammatory cytokine- and ROS-producing to M2 iNOS−/CD206+ anti-inflammatory and debris clearing. Effective clearance of myelin and cellular debris is a prerequisite for terminal differentiation of oligodendrocyte progenitor cells into myelin-producing mature oligodendrocytes, and a lack of microglia prevents normal remyelination. The present study reveals that both Iba1+ area and the fraction of iNOS-expressing Iba1+ cells decreased following VNS, indicating that VNS reduces the proinflammatory state of innate immune cells in the CNS. In addition, the shift in these cells to a CD206+ resolution phenotype following VNS indicates the induction of an accelerated repair response, which correlates to the observed shortened duration of EAE symptomatology.

Neutrophil infiltration has been implicated in MS and EAE pathogenesis and reduction in infiltration may ameliorate disease. Quantitative neutrophil data from the most involved immunofluorescence fields of view (FOVs) demonstrated that VNS significantly decreased the number of infiltrating neutrophils when assessed at 0-2 DPSO. This finding is consistent with VNS- and α7nAChR-induced restriction of neutrophils.

Dysfunction in the acetylcholine neurotransmitter system has been reported and strategies that enhance cholinergic transmission have been demonstrated to ameliorate both MS and EAE. Specific or non-specific α7nAChR agonism (e.g. with nicotine, galantamine) results in suppression of neuroinflammation and symptoms of disease in numerous models of EAE and MS. In addition, treatment with the DMT dimethyl fumarate, but not IFN-β, enhanced cholinergic transmission in MS patients. Here, we reported that VNS increased SC α7nAChR gene expression. In addition, genes encoding β2AR and ChAT, other key molecular conductors of the inflammatory reflex, were also acutely increased by VNS. Intriguingly, preliminary data generated from a small pilot chronic study investigating the durability of VNS in EAE demonstrated that splenocytes and isolated splenic T-cells harvested from VNS-treated rats showed a 1.6-2.4× increase in gene expression of Adrb2, Chat, and Charna7 after five months of daily stimulation compared with Sham and naïve rats. These data support the hypothesis that chronic VNS can increase expression of key inflammatory reflex genes in early EAE, as well as over a longer-term course of treatment, without indication of reflex tachyphylaxis.

Increased expression of Mbp, Mog, and Plp is required for oligodendrocyte progenitor (OPC) differentiation, with OPC maturation in turn required for remyelination. It is of interest that VNS also differentially regulates expression of genes encoding myelin sheath components and transcription factors that are involved in myelin synthesis and oligodendrocyte differentiation. VNS significantly upregulated expression of Mbp, Mog, Plp, as well as transcriptional regulators Sp1, Sox 10, and Pur alpha at 0-2 DPSO. Intriguingly, activation of nAChRs on OPCs has been shown to upregulate these genes and induce rapid differentiation into mature oligodendrocytes. Independently, a group has reported that VNS increased the rate of remyelination in the corpus callosum of rats injected with lysolecithin, while a second group has reported that upon secession of a cuprizone demyelination diet, VNS not only accelerated remyelination in the motor cortex of mice, but also significantly increased the extent to which remyelination occurred. Taken together, these data suggest not simply an immunoregulatory role for VNS, but perhaps a reparative role as well; a potential for VNS treatment to enhance remyelination in MS.

In addition to the inflammatory reflex, another mechanism through which VNS may positively impact inflammation, leukocyte infiltration, BBB integrity and macrophage/microglia phenotype is by increasing specialized pro-resolving mediators (SPMs), lipid classes that orchestrate inflammation and its resolution. Vagotomy reduces SPMs, inhibits normal resolution, and results in exacerbation of disease, while VNS enhances resolution of inflammation and subsequent tissue damage via the production of endogenous SPMs. The VN itself stores SPMs and releases them upon stimulation to reduce both proinflammatory prostaglandins and leukotrienes. Recent data show a disruption in typical homeostatic patterns of SPMs in patients with MS and treatment of isolated human brain endothelial cells and monocytes from MS patients with relevant SPMs enhances BBB integrity and reduces both monocytic inflammatory responses and transendothelial migration. Furthermore, treatment with either resolvin D1 or maresin-1 ameliorated disease in mouse models of EAE.

There are several limitations to this study. While it is highly advantageous to use a fully implanted system to autonomously stimulate awake animals without the stresses of daily human interaction with immobilization and infection-prone percutaneous leads, the complete system size restricted our ability to study EAE in mice. The widely used Lewis rat model produces a brief monophasic disease course which limited the window for therapeutic intervention. Therefore, the experimental design was neither purely therapeutic nor preventative, rather a hybrid of the two (a “semi-established” disease model with treatment initiation during concurrent subclinical CNS inflammation). In addition, this rat model does not exhibit robust CNS demyelination, so the ability to fully assess the extent of myelin protection by the therapy was limited. Additionally, only female animals were studied and independent animal sample sizes for secondary endpoints were relatively small.

These results of VNS in rat EAE reveals therapeutic effects comparable to the MS drug teriflunomide at allosteric equivalents 2.1-4.1 fold greater than those approved for human use, as well as amelioration of pathophysiologic drivers and correlates of disease. The high lifetime incidence (50%) of depression in MS, together with demonstrated VNS efficacy in depression, supports that VNS may represent a safe and effective long term therapy that may address not only MS but also a mood disorder comorbidity. Perhaps the most provocative findings are that VNS can shift microglial phenotype to promote clearance and repair and upregulate the expression of genes involved in myelin synthesis, indicating that VNS may not only ameliorate inflammation and clinical symptoms of disease but also enhance myelin formation and repair. Additional animal model studies are currently underway to further investigate the capacity of VNS to independently promote remyelination.

### Materials and Methods

In regard to FIGS. 12A to 19D, the following materials and methods were used. Five to six weeks old female Lewis rats (Charles River Laboratories, location) were housed in the Center for Comparative Physiology (CCP) facility, Feinstein Institute for Medical Research in temperature- and humidity-controlled rooms on a 12 h dark/12 h light cycle, with rat chow and water provided ad libitum. All experimental procedures involving animals were preapproved by the Institutional Animal Care and Use Committee (IACUC) and carried out in accordance with NIH guidelines for the Care and Use of Laboratory animals and the IACUC.

Stimulation device implantation: The subacute to chronic implantation methodology was adapted from well-developed principles and techniques. Rats were anesthetized with isoflurane (3% induction, 2-2.5% maintenance) and incision sites prepared by hair removal and disinfection (povidone iodine solution and 70% isopropyl alcohol). Rats were placed supine and the left vagus nerve isolated through a 1 cm medial incision. Rats were then placed prone and a 1.5-2 cm incision made on the dorsal skin. A pocket to receive the stimulation device was created using blunt scissors by separating skin from underlying tissue. A subcutaneous tunnel for the leads was formed between the pocket and the incision in the neck. The device (FIG. S1) was positioned under the skin on the dorsal side and secured with non-absorbable nylon suture. The electrode cuff was gently pulled through the tunnel and secured around the left vagus nerve. Sham stimulated rats were implanted in the same way, but with the sham device lacking a terminal electrode cuff. Incisions were closed with surgical staples (Stoelting™ EZ Clip wound closure kit, Wood Dale, IL). Rats were given local anesthetic (Bupivacaine, 0.5%, s.c., Hospira Inc, Lake Forest, IL, s.c., Manufacturer), analgesia (Buprenorphine 0.03 mg/kg, sc., Par Pharmaceutical, Chestnut Ridge, NY), antibiotics (Baytril 10 mg/kg, sc., Bayer, Berlin), and resuscitation fluids (4-5 ml of warm normal saline, s.c, SAI Infusion Technologies, Lake Villa, IL) and then placed on a heating pad until sternal recumbency was regained. Analgesia (Meloxicam infused diet gel, 1.5 mg/ml, Covetrus, Portland, ME) was also provided for three days post-surgery.

EAE induction and VNS stimulation: Six to seven days after nerve stimulator device implantation and while acutely under anesthesia (isoflurane, 3%), EAE was induced by injection (s.c) of emulsified peptide from guinea pig myelin basic protein (gpMBP69-88, 100 mg/rat+ complete Freund adjuvant; Hooke laboratories, Lawrence, MA) into both dorsal hind leg flanks (100 ml/side). All surgical staples from device implantation were also removed at this time. Seven days after immunization (DPI; the approximate day of EAE symptom onset), daily electrical vagus nerve stimulation (750 μA, 10 Hz, 60 s, 0.25 ms pulse width, TID) was initiated and continued through 21 DPI or day of euthanasia. Rats in the disease control group were naïve to manipulation prior to EAE induction.

Treatment of EAE with teriflunomide: EAE was induced as above in rats that did not undergo implantation procedures. Teriflunomide stock; 50 mg of teriflunomide (Cat #5069/50, R&D System (Tocris), Minneapolis, MN) dissolved in 2 ml of 0.5% DMSO (Cat #D2650, Sigma, St. Louis, MO) and mixed with 2 ml of carboxymethyl cellulose (Across Organics, Cat #AC332601000, Thermo Fisher Scientific, Fair Lawn, NJ) dissolved in 25 mM Tris-HCl pH 7.5 (Cat #15567-027, Invitrogen, Carlsbad, CA) was prepared and diluted into fresh working solution with 25 mM Tris-HCl after the rats were weighed that day. Each rat was administered 0.2 ml teriflunomide (1 mg/kg/day or 3 mg/kg/day) or control (25 mM Tris-HCl) by oral gavage, from 7 to 21 DPI.

Body weight and clinical score recording: Clinical scores based on observable disease symptoms (from 0-no symptoms) to 5-moribund or death) were recorded in a blinded manner, according to a standard scoring guideline (Hooke laboratories, Lawrence, MA). Body weights were recorded from 0-21 DPI.

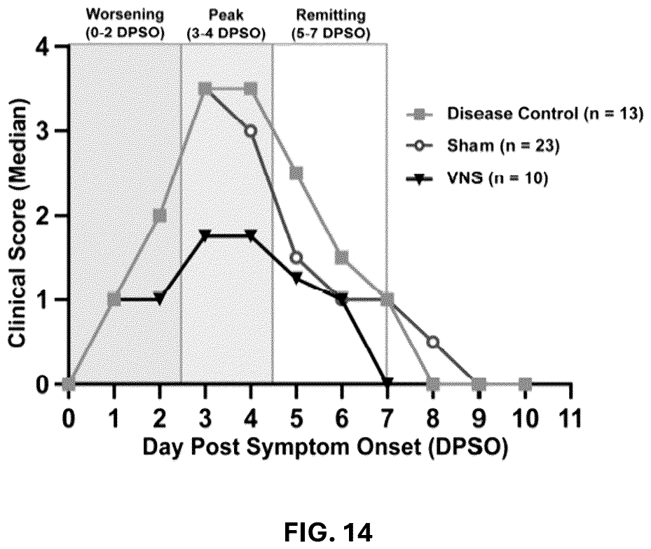

Spinal cord samples collection: EAE rat spinal cord samples were collected at 0-2 (worsening), 3-4 (peak), and 5-7 (remitting) days post symptom onset (DPSO; FIG. 14). Rats were perfused with approximately 150-200 ml of ice-cold PBS via cardiac puncture under deep anesthesia, and SCs were collected for flow cytometry analysis. A small SC segment close to the lumbar region was collected and stored in RNA later solution (Invitrogen, Cat #AM7020) for qPCR analysis. A section of lumbar spinal cord was dissected and stored in 4% paraformaldehyde (PFA) (Electron Microscopy Sciences, Hatfield, PA) solution for IHC analysis.

Histochemistry: Paraffin-embedded lumbar spinal cord blocks were stained with luxol fast blue (LFB) and nuclear fast red or hematoxylin and cosin (H&E) to visualize demyelination and cellular infiltrates, respectively. Briefly, decalcified lumbar SC sections were serially dehydrated in ethanol. Then the samples were cleared in xylene and paraffinized at 60° C. for 1 hour. Paraffin sections (3 and 8 mm) were prepared and utilized for histochemical and immunofluorescence staining, respectively. H&E staining: Sections were deparaffinized and hydrated following standard procedure then immersed in hematoxylin solution for 4 min and washed in running tap water for 15 min. Slides were then incubated with cosin solution for 2 min and differentiated in 70% ethanol. Following differentiation, slides were dehydrated in ethanol, cleared in xylene and mounted with Eukitt quick-hardening mounting medium (Sigma-Aldrich, St. Louis, MO).

Luxol Fast Blue (LFB) and Nuclear Fast Red staining: Lumbar SC sections (3 mm) were deparaffinized and hydrated following standard procedure and incubated with LFB solution (0.1 g LFB, 100 ml 95% ethanol, and 0.5 ml 10% acetic acid) overnight at 58° C. Slides were washed in running tap water for 10 min. Slides were differentiated with 0.05% lithium carbonate solution for 10 to 20 seconds followed by differentiation with 70% ethanol until gray and white matter were distinguished. Slides were then washed in distilled water and immersed in fast red solution (IHC World LLC, Ellicott City, MD) for 5 min, washed immediately in distilled water to remove excess staining. Slides were then dehydrated in absolute ethanol, cleared with xylene, and mounted with Eukitt quick-hardening mounting medium (Sigma-Aldrich, St. Louis, MO).

Immunofluorescence staining: 8 μm spinal cord sections were deparaffinized and antigen retrieval was performed using antigen unmasking solution (H-3300; Vector labs, Newark, CA) in a steamer for 10 min. Sections were cooled at room temperature and washed 3×5 min in PBS. Sections were permeabilized with PBS containing 0.3% Triton-X for 30 min at room temperature. Sections were washed in PBS for 3×5 min, then blocked with 4% normal goat serum and 4% normal donkey serum for 45-60 min at room temperature. Sections were incubated with respective primary antibodies such as Alexa-488 conjugated mouse anti-rat CD4 (domain 1, Cat #MCA55A488, Biorad, Hercules, CA); eFluor660 conjugated mouse anti-rat IFNg (clone DB1, Cat #50-7310-80, Invitrogen, Carlsbad, CA); goat anti-IL-17 (Cat #SC-6078, Dallas, TX); sheep anti-fibrinogen (Cat #F4203-02F, US Biologicals, Salem, MA); Alexa-488 conjugated anti-GFAP (Cat #53-9892-82, Invitrogen, Carlsbad, CA), Alexa-488 conjugated claudin-5 (Cat #352588, Thermo Fisher Scientific, Waltham, MA), rabbit anti-CD31 (Cat #PA5-32321, Thermo Fisher Scientific, Waltham, MA); mouse anti-Iba1 antibody (Cat #ab283319, Abcam, Waltham, MA); Alexa-568 conjugated rabbit anti-iNOS (Cat #Ab20995, Abcam, Waltham, MA); Alexa-647 conjugated rabbit anti-CD206 (Cat #Ab195192, Abcam, Waltham, MA); Alexa-647 conjugated rabbit anti-CD31 (Cat #Ab218582, Abcam, Waltham, MA); and mouse anti-CEACAM8/CD66b (Cat #NB100-77808, Novus Biological LLC, Centennial, CO) at 1:100 concentration in 1% normal goat serum at 4° C. overnight. Sections incubated with unconjugated antibodies were fluorescently labelled with respective secondary antibodies and incubated for 2 h at room temperature. Sections were then washed and mounted with DAPI containing mounting medium (Fluoroshield, Cat #F0657, Sigma, St. Louis, MO). Images were taken cither using a KEYENCE BZ-X800 or Zeiss LSM 900 confocal microscope.

SC sample preparation for flow cytometry: SC samples were minced separately using surgical blades in the presence of 6 ml digestion mix (1 mg/ml collagenase D, 50 μg/ml of DNase-I) and incubated at 37° C. for 30 to 40 minutes in a rotating apparatus. After digestion, the tissue samples were gently filtered through a 70 mm mesh strainer. 10 ml of PBS was added, the tube centrifuged at 2000 rpm for 5 min at 4° C., and the cell pellet reserved for flow analysis. To eliminate myelin debris from SC samples, the digested cell suspension was gently layered onto 6 ml of 15% BSA in PBS in a 15 ml conical tube and spun for 15 min at 1900 rpm at 4° C., without applying the brake in the centrifuge. Separated myelin debris was carefully aspirated from above the cell pellet of unmyelinated glia and lymphocytes that had settled at the bottom of the tube. The pellet was washed with ice-cold PBS and used for fluorochrome staining.

Flow cytometry analysis: Isolated SC cells were divided equally into two tubes (approximately 2×106 cells per tube), one serving as unstained control and the second for antibody staining. Tubes were incubated with 100 ml of FACS buffer (2% FBS in PBS and 1 mM EDTA) in a round bottom 96 well plate on ice. Staining cell samples were incubated with mouse anti-rat CD32 antibody (Cat #550270; BD Biosciences, Franklin Lakes, NJ) (1 ml/well/100 ml FACS buffer) on ice for 30 min to block non-specific binding, then incubated with Aqua live/dead fluorochrome (0.25 ml/well) for 30 minutes on ice and washed with FACS buffer. Cells were then resuspended in FACS buffer and incubated with 0.5 ml/well of the respective fluorochrome-conjugated antibodies such as pacific blue conjugated mouse anti-rat CD45 (Cat #202226, San Diego, CA); Alexa-488 conjugated mouse anti-rat CD4 (domain 1, Cat #MCA55A488, Biorad, Hercules, CA) and incubated for 30 additional minutes on ice. Cells were washed with ice-cold FACS buffer (200 ml/well) and centrifuged at 2000 rpm for 3 min at 4° C. Cells were resuspended in 25 ml of FACS buffer before being mixed with 100 ml of Cytofix/Cytoperm (Franklin Lakes, NJ) solution and incubated for 20 min at 4° C. Cells were then washed two times with Permwash buffer (Franklin Lakes, NJ) (hereafter, we keep the cells for the rest of the procedures in Permwash buffer) and incubated with cytosolic antibodies such as PE-eFluor610 conjugated mouse anti-rat IL-17 (clone eBio1787, Cat #61-7177-82, Invitrogen, Carlsbad, CA); eFluor660 conjugated mouse anti-rat IFNg antibody (clone DB1, Cat #50-7310-80, Invitrogen, Carlsbad, CA) for 30 min on ice. After incubation, cells were again washed with Permwash buffer twice and filtered through Falcon filtertop flow cytometry tubes (Cat #352235) to remove any debris prior to flow cytometry. Compensation control: Compensation controls for each color were set using both negative and antibody binding positive beads (BD Biosciences, Franklin Lakes, NJ) following manufacturer instructions. Briefly, in each tube, one drop of each kind of bead was added and incubated with and without the conjugated antibodies (one color per tube) and incubated at room temperature for 15 to 20 min. Beads were then washed with FACS buffer and used as compensation controls for each fluorochrome. Fluorescein minus one (FMO) control: To validate the spillover effect of each fluorochrome into other spectra, we performed an FMO control. Blood was collected from EAE rat, and the red-blood cells were lysed with ACK lysis buffer (Cat #A1049201, Fisher Scientific, Biochemia, NY). 4×104 blood cells were incubated with the cocktail of all fluorochromes in the same tube, except one. We followed the same strategy for each fluorochrome in their respective tubes to measure spillover, if any, and 2×104 cell samples were gated in the BD FACSymphony A3 cell analyzer (Franklin Lakes, NJ).

qPCR assay: Following the Trizol (Life technologist, Grand Island, NY) nucleic acid purification method, total RNA was isolated from SC samples. cDNA was synthesized using Super Script VILO cDNA kit (Invitrogen, Carlsbad, CA), following manufacturer instructions. Quantitative real time PCR was performed using Power-up SYBR green master mix solution in the Roche480 thermal cycler, for Th1 (Ifng, Il112) and Th17 (Il17) associated genes, key components of the inflammatory reflex (Adrb2, Chat and Charnα7), and myelin synthesis-related genes (Mbp, Mog, Plp), and known transcription factors that bind at the promoter region of the Mbp gene (Sp1, Sox 10 and Pur alpha) (96). Primers to known sequences were used.

Each sample was analyzed in duplicate, with each experiment repeated at least twice. Gene expression was presented as relative fold change over control gene (Actin beta) calculated based on the DDCT values. The relative expression of analyte to Actin beta gene was normalized to the mean expression of the Sham group.

### Quantification of Inflammation and Demyelination:

The inflammatory lesion area of the rat lumber spinal (at least two sections from each rat; approximately 0.5 cm apart) were calculated in both white and gray matter regions using ImageJ (NIH). To restrict total area to definite lesions, only lesions with areas larger than the 25th percentile (3000 μm2) were tabulated and normalized to mean Sham lesion area. Demyelination was quantified in at least 2 sections from each rat; approximately 0.5 cm apart. The loss of LFB staining in the white matter region was tabulated by manually drawing outlines around each demyelinated region using ImageJ. The total areas of inflammatory lesions and demyelination were normalized to Sham and calculated from at least 3 rats per group.

### Immunofluorescence Analysis:

For measurement of GFAP intensity, ImageJ was used to outline the entire spinal cord and the mean fluorescence intensity (MFI) within the outlined structure was recorded for statistical analysis. At least 2 spinal cord sections were analyzed per animal, and all animals were normalized to Sham.

Fibrinogen deposition into the white and gray matter regions of the SCs was quantified using ImageJ, following a previously published method measuring myelinated nerve fibers. Briefly, the images (most involved fields of view; FOV; 0.10 mm2) were converted into black and white at a constant intensity threshold, and the image dimension were calibrated to a known scale. Total pixel intensity was measured and the integrated pixel intensity (arbitrary units) over the surface area of the image was calculated and normalized to the mean Sham. To compare the number of infiltrating neutrophils among naïve, Sham and VSN rats, CD66b+ cells were manually counted from at least 2 sections per animal and 4-7 FOVs (0.10 mm2) per section.

For microglia/macrophage analysis in both lesion and non-lesion areas of the Sham and VNS rat spinal cords, 4-11 sections with approximately were analyzed per animal. To measure the area fraction occupied by microglia/macrophage, images were binarized using an automated intensity threshold and the percentage thresholded pixels was reported as Iba1+ area fraction. To assess microglial/macrophage states, microglial/macrophage expression of iNOS vs. CD206 was quantified as putative “M1” vs. “M2” state, respectively. iNOS and CD206 images were thresholded and binarized. The overlap between microglia/macrophage and either CD206 or iNOS was measured by multiplying the binarized Iba1 with either CD206 or iNOS images. The percentage of CD206- or iNOS-expressing microglia was calculated by dividing the number of colocalized signal pixels (Iba1+CD206+ or Iba1+ iNOS+) by the total Iba1+ pixels. The ratio between iNOS-expressing and CD206-expressing microglia was subsequently calculated.

### Statistical Analysis:

Clinical scores are plotted as median±IQR and Kruskal-Wallis test was used to compare medians on individual days. The complete curves were analyzed by 2-way mixed model ANOVA with treatment and time (DPSO) as independent variables, with multiple comparisons with Dunnett's post hoc test to compare means of Sham and Disease Control to mean of VNS on individual days. The AUC, Maximum clinical score, and number of symptomatic days were first analyzed for statistical outliers (ROUT; Q=1%) and compared to the VNS group by ANOVA followed by Tukey's multiple comparison test.

All other data are presented as the mean±SEM. Quantification of inflammation, demyelination, and quantitative immunofluorescence targets between VNS and Sham groups were analyzed by 2-tailed unpaired Student's t-test or by ANOVA followed by Tukey's multiple comparison test, as appropriate. Infiltration of CD45+/CD4+/IL17+ and CD45+/CD4+/IFN-γ+ cells by flow cytometry are reported as a fraction of CD45+ cells and normalized to Sham. Differences in mean between VNS and Sham groups were calculated by unpaired 2-tailed t-test with Welch's correction.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating multiple sclerosis (MS), the method comprising:
receiving, in an implanted vagus nerve stimulator implanted in a patient, a command to apply a remyelinating stimulation to a patient; and
applying electrical stimulation of between about 0.25 mA and about 5 mA at a duty cycle of less than 10 percent to the patient's vagus nerve from the implanted vagus nerve stimulator to reverse demyelination and increase remyelination of the patient's nerves to treat the patient's MS.

2. The method of claim 1, wherein applying comprises applying the electrical stimulation, followed by an off-time of at least 10 minutes.

3. The method of claim 1, wherein applying comprises applying the electrical stimulation for less than about 2 minutes.

4. The method of claim 1, wherein applying comprises applying the electrical stimulation for less than about 2 minutes, followed by an off-time of at least 10 minutes.

5. The method of claim 1, further comprising continuously monitoring the patient for the one or more biomarkers for MS.

6. The method of claim 1, further comprising monitoring one or more biomarkers for demyelination.

7. The method of claim 6, wherein the one or more biomarkers for demyelination is selected from the group consisting of neurofilament, glial fibrillary acidic protein, the monocyte macrophage marker CD163, the glial activation marker YKL-40, the B cell chemoattractant CXCL13, miRNA, mRNA, myelin reactive t cells, Kir4.1 antibodies, osteopontin, and microbiome associated lipopeptides.

8. The method of claim 1, further comprising repeatedly applying a low duty-cycle electrical stimulation of between about 0.25 and about 5 mA to the patient's vagus nerve for less than 2 minutes, followed by an off-time of between about 12 and about 48 hours.

9. The method of claim 1, further comprising concurrently treating the patient with a pharmacological agent to treat demyelination.

10. A method of treating MS by reversing demyelination in a patient having MS, the method comprising:
receiving, in an implanted vagus nerve stimulator in a patient diagnosed with MS, a command to apply a remyelinating stimulation to the patient; and
applying electrical stimulation of between about 0.25 mA and about 5 mA at a duty cycle of less than 10 percent to the patient's vagus nerve from the implanted vagus nerve stimulator to reverse demyelination and increase remyelination of the patient's nerves.

11. The method of claim 10, wherein applying comprises applying the electrical stimulation, followed by an off-time of at least 10 minutes.

12. The method of claim 10, wherein applying comprises applying the electrical stimulation for less than about 2 minutes.

13. The method of claim 10, wherein applying comprises applying the electrical stimulation for less than about 2 minutes, followed by an off-time of at least 10 minutes.

14. The method of claim 10, further comprising monitoring the patient for the one or more biomarkers for demyelination.

15. The method of claim 14, wherein the one or more biomarkers for demyelination is selected from the group consisting of neurofilament, glial fibrillary acidic protein, the monocyte macrophage marker CD163, the glial activation marker YKL-40, the B cell chemoattractant CXCL13, miRNA, mRNA, myelin reactive t cells, Kir4.1 antibodies, osteopontin, and microbiome associated lipopeptides.

16. The method of claim 10, further comprising repeatedly applying a low duty-cycle electrical stimulation of between about 0.25 and about 5 mA to the patient's vagus nerve for less than 2 minutes, followed by an off-time of between about 12 and about 48 hours.
